(19) United States
(12) Patent Application Publication
(10) Pub. No.: US 2005/0142547 A1
(43) Pub. Date: Jun. 30, 2005

(54) Diagnosis of vascular disease susceptibility using bacteriophage phi-cpn1 host chlamydia

(76) Inventors: Robert Brunham, Vancouver (CA); Karuna P. Karunakaran, Vancouver (CA); Jamie Blanchard, Winnepeg (CA)

Correspondence Address: MCKENNA LONG & ALDRIDGE LLP, 1900 K STREET, NW, WASHINGTON, DC, 20006, US

(21) Appl. No.: 10/478,677

(22) PCT Filed: May 23, 2002
(86) PCT No.: PCT/CA02/00730

Related U.S. Application Data
(60) Provisional application No. 60/293,373, filed on May 23, 2001.

(51) Int. Cl. C12Q 1/68

(52) U.S. Cl. 435/6

(57) ABSTRACT

Methods are provided for determining susceptibility of a patient to vascular disease according to presence of chlamydia infected with bacteriophage ΦCpn1, and compositions for use in such methods. The compositions include bacteriophage ΦCpn1 proteins and nucleic acids, elementary bodies from chlamydia infected with ΦCpn1, and antibodies thereto.

### FIELD OF THE INVENTION

This invention relates to diagnosis and treatment of vascular disease.

### BACKGROUND OF THE INVENTION

The etiopathology of abdominal aortic aneurysm (AAA) is still largely unknown. It has been postulated that localized inflammation and destruction of structural connective tissue by a direct infectious or autoimmune process is involved. There is also some evidence of a possible association between Chlamydia infection and vascular disease (including atherosclerosis and AAA). Animal studies have demonstrated that following inoculation in the respiratory tract, C. pneumoniae disseminates throughout the vascular system, and in some animal models (such as models for hypercholesterolemic conditions) may result in the development of atherosclerotic lesions [1-6]. There is some suggestion that antibiotic therapy might lower the risk of acute coronary events [7, 8]. Observational studies have also shown some relationship between the presence of C. pneumoniae and AAA [9, 10]. Most people will be infected by C. pneumoniae by the age at which the clinical manifestations of atherosclerosis usually appear [11] and some argue that C. pneumoniae is simply an organism that more readily infects diseased arteries [12], or that its association with vascular disease is confounded by its association with other atherosclerotic risk factors [13].

Further complicating the situation was the apparent genetically-linked variability of host response to genital Chlamydia trachomatis infection as manifested by differences in risk of development of pelvic inflammatory disease [14]. Variability in host response could be why some persons are more prone to develop vascular disease when infected with C. pneumoniae. There could also be variations in the vasculotrophism and pathogenicity of different C. pneumoniae strains.

DNA sequencing has shown genetic variation in C. pneumoniae [15-17]. One C. pneumoniae strain (AR39) exhibits a 4524 nucleotide single-stranded DNA bacteriophage ΦCpn1 [16]. Phage-bearing strains of some bacteria have been found to be more pathogenic than phage-free strains [18] but the implications of phage presence in Chlamydia was unknown.

### SUMMARY OF THE INVENTION

It has now been discovered that C. pneumoniae containing the phage ΦCpn1 is involved in vascular disease.

This invention provides a method for identifying a subject susceptible to vascular disease, comprising: i) providing a sample from a subject to be tested for susceptibility to vascular disease; and ii) detecting the presence or absence of a bacteriophage ΦCpn1 host Chlamydia component in the sample, or an antibody to said component, wherein presence of said component or antibody is indicative of the presence of said host Chlaniydia in the subject and susceptibility of the subject to vascular disease.

This invention provides methods to determine whether a patient is susceptible to vascular disease, including abdominal aortic aneurysm (AAA), atherosclerosis, stroke, heart attack, etc. Such a patient could be one with a predisposition or known risk factor to vascular disease (such as high blood lipid levels or smoking) but the methods of this invention determine susceptibility independent of such known risk factors or predispositions and are predictive without prior assessment of such risk factors or predispositions.

The methods of this invention may comprise providing a sample of biological material from a patient to be tested. Such biological material may be a tissue sample, fluid sample (e.g. blood or serum sample) or sample of material associated with the body such as sputum.

In the methods of this invention, a sample is tested for the presence in the patient of host C. pneumoniae which contains a bacteriophage. The host C. pneumoniae which may be detected by such testing may be strain AR39.

This invention may be performed by directly detecting the presence of a host Chlamydia such as AR39. Such an embodiment may be used when it is unknown if a patient has a known risk factor or predisposition. This method is useful even if it is unknown if a subject has elevated lipid levels or does not have a hypercholesterolemic condition. Detection in these embodiments may be by means of antibodies specific to AR39 such as those described herein. Such antibodies may be specific to AR39 elementary bodies.

Methods of this invention preferably involve direct detection of the phage ΦCpn1 alone or in combination with direct detection of presence of the host. While the presence of the phage is indicative of the presence of host C. pneumoniae containing the phage, directly testing for the phage has the best correlation to susceptibility to vascular disease. In such embodiments, the detection of phage may be detection of the phage itself, of a phage peptide, or nucleic acid associated with the phage, or of an antibody to a phage peptide that is present in the patient. Known methods may be employed, such as obtaining a nucleic acid from a fraction taken from a sample of bodily fluid from a patient and determining the presence or absence of part or all of a bacteriophage genome.

This invention provides an antibody which binds to a phage antigen such as the peptides of SEQ ID NO:8-13.

This invention also provides antibodies to phage ΦCpn1 which are suitable for use in the aforementioned methods. Such methods for producing such antibodies may be specific to a peptide associated with the phage. Preferably, such an antibody will be specific to an antigenic epitope present on the surface of the phage, which includes the antibodies to phage capsid proteins and those described herein for protein Vp1. Alternatively, antibodies for use in the aforementioned methods may be anti-idiotypic antibodies in that they are specific for antibodies to the phage or a phage peptide.

This invention also provides compositions comprising one or more peptides associated with phage ΦCpn1 for eliciting an immune response against phage ΦCpn1 (or a peptide of said phage). Also provided are nucleic acid compositions and methods of using such compositions for expressing phage peptides and for eliciting an immune response by expression of the nucleic acid in mammalian subjects to which the composition is administered, wherein the composition comprises one or more nucleic acids encoding all or part of a peptide corresponding to a peptide of phage ΦCpn1. These compositions may comprise pharmaceutically acceptable diluents, excipients, and adjuvants to facilitate administration of the composition to a patient or animal and in the case of the nucleic acid composition, may comprise a vector for expression of the nucleic acid in a subject.

The invention also provides the use of all or part of a phage ΦCpn1 peptide in an immunoassay to test for the presence of phage or anti-phage antibodies in a biological sample.

This invention provides a kit for use in the method of claim 1, comprising in a commercial package: i) one or more detecting moieties selected from the group consisting of: a ΦCpn1 peptide, or peptide having substantial sequence identity to a peptide or fragment thereof; an antibody that binds to a ΦCpn1 peptide; one or more oligonucleotides complementary to a ΦCpn1 nucleic acid; an antibody that binds to Chlamydia AR39 elementary bodies; and, a peptide that binds to an antibody that binds to such elementary bodies; and, ii) instructions for use of the one or more detecting moieties for detecting the presence of a bacteriophage ΦCpn1 host Chlamydia components in a sample.

This invention also provides methods for selecting a course of treatment of (or for treatment of) a patient who is or may become susceptible to vascular disease (or which patient is or may become infected with C. pneumoniae comprising the bacteriophage). The method may comprise administering an agent effective against the C. pneumoniae such as an antibiotic.

In this specification, the term “peptide” is meant to encompass a peptide of any length including moieties that may be referred to as polypeptides and proteins.

### BRIEF DESCRIPTION OF THE DRAWINGS

FIG. 1: shows differentiation of phage-containing (AR39) and phage-free (CWL029) strains of C. pneumoniae using PCR (A), RT-PCR (13) or Western blotting (C).
A). PCR analysis of DNA isolated from HL cells infected with C. pneumoniae strain AR39 (lanes 1 & 3) or CWL029 (lanes 2 & 4) using primers specific to chlamydial 16s RNA (lanes 1 & 2) or ΦCpn1 vpl gene (lanes 3 & 4).
B). RT-PCR analysis of total RNA isolated from HL cells infected with C. pneumoniae strain AR39 (lanes 1, 3, & 5) or CWL029 (lanes 2, 4, & 6) using primers specific to chlamydial 16s RNA (lanes 1-4) or ΦCpn1 vpl gene (lanes 5 & 6). Lanes 3 & 4 are similar to lanes 1 & 2 except that the template was total RNA instead of reverse transcribed cDNA.
C). Western blot analysis of C. pneumoniae strains AR39 (lane 2) and CWL 029 (lane 1). Boiled samples of purified AR39 and CWL 029 elementary bodies were subjected to electrophoresis in 10% SDS-PAGE gels and the blotted nitrocellulose membranes were incubated with polyclonal anti-Vp1 antibodies followed by incubation with alkaline phosphatase conjugated secondary antibodies and colour detection.

FIG. 2: shows C. pneumoniae strain typing. DNA isolated from 3 laboratory strains [AR39 (Lane 1), CWL029 (Lane 2), CM1 (Lane 3)] and 6 clinical isolates [K7 (Lane 4), Bay 13 (Lane 5), Bay 16 (Lane 6), Bay 19 (Lane 7), Bay 20 (Lane 8), and Bay 21 (Lane 9)] were PCR analysed using specific primers for the phage vpl gene (Panel A), C. pneumoniae 16s RNA (Panel B), or C. pneumoniae ORF CP0543 (Panel C). ORF CP0543 represents the truncated version of a ΦCpn1 ORF found in C. pneumoniae genome (24) and used here as an additional control to the 16s RNA.

### DETAILED DESCRIPTION OF THE INVENTION

The methods of this invention involve detection of the presence of bacteriophage contained in C. pneumoniae, such host Chlamydia, or antibodies to the phage. The presence of bacteriophage may, for example, be determined by obtaining nucleic acids from C. pneumoniae (e.g. as present in a biological sample from a patient) and determining (e.g. by sequencing) the presence of a phage genome (for example, see numbered reference [16]). The detection may be of Chlamydia strain AR39, such as by the methodology described herein. Preferably, the detecting will be specific to the presence of phage ΦCpn1.

Detection of phage ΦCpn1 may be preformed using general methodologies known in the art such as those applied and described below. One methodology is to detect the presence of phage specific peptides. These peptides may be detected by isolating proteinaceous material from a biological sample and determining the sequence of peptides so isolated and comparing to the known sequence of phage proteins as described below. Preferably, such detecting will make use of an intermediate agent such as an antibody specific for the phage peptide. A preferred phage protein is Vp1 as employed in the examples below. The sequence of Vp1 is described below in Table 3.

Antibodies to phage peptides may be prepared by a variety of known methods. Such antibodies may be polyclonal, monoclonal, or may be fragments of antibodies which bind to the antigen.

For the production of antibodies to phage, phage peptides, or host Chlamydia as antigen, various mammals including goats, rabbits, rats, mice, humans, and others, may be immunized by injection with the antigen. Depending on the species, various adjuvants may be used to increase immunological response. Such adjuvants include, but are not limited to, Freund's, mineral gels such as aluminum hydroxide, and surface active X substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

It is preferred that immunogenic peptides used to induce antibodies have an amino acid sequence consisting of at least five amino acids, and more preferably at least 10 amino acids. It is also preferable that they are identical to a portion of the amino acid sequence of the natural protein, and they may contain the entire amino acid sequence of the phage peptide. Short stretches of phage amino acids may be fused with those of another protein such as keyhole limpet hemocyanin and antibody produced against the chimeric molecule.

Peptides corresponding to a phage amino acid sequence may be synthesized using methods known in the art, including direct synthesis or the recombinant methods disclosed in the examples below. Such peptides may also be made to incorporate a N-terminal moiety such as cysteine to facilitate conjugation to other molecules (e.g. to enhance immunogenicity). Conjugation of cysteine may be mediated by an agent such as m-maleimidobenzoyl-N-hydroxy-succinimide ester (MBS). Antibodies that specifically react with the peptide may be purified from the antisera by affinity chromatography, for example by using Cellulofine™ (Seikagaku Corporation) conjugated with the peptide. The resulting antibodies may be tested by immunoblotting.

Monoclonal antibodies to phage peptides or anti-idiotypic monoclonal antibodies may be prepared using any of the techniques known in the art which provide for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; Cole, S. P. et al. (1984) Mol. Cell Biol. 62:109-120).

One process for obtaining the hybridomas of this invention involves starting from spleen cells of an animal, e.g. mouse or rat, previously immunized in vivo or from spleen cells of such animals previously immunized in vitro with an antigen and fusing the immunized cells with myeloma cells under hybridoma-forming conditions; and selecting those hybridomas which secrete the monoclonal antibodies which are capable of specifically recognizing the phage peptide.

Selected hybridomas are cultured in appropriate culture medium; and then the secreted monoclonal antibodies are recovered; or alternatively the selected hybridoma is implanted into the peritoneum of a mouse and, when ascites has been produced in the animal; the monoclonal antibodies formed from the ascites are recovered. Monoclonal antibodies of the invention may be prepared by conventional in vitro techniques such as the culturing of immobilized cells using e.g. hollow fibers or microcapsules or such as the culturing of cells in homogeneous suspension using e.g. airlift reactors or stirred bioreactors.

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452454). Alternatively, techniques described for the production of single chain antibodies may be adapted, using methods known in the art, to produce phage-specific single chain antibodies. Antibodies with related specificity, but of distinct idiotypic composition, may be generated by chain shuffling from random combinatorial immunoglobulin libraries (Burton D. R. (1991) Proc. Natl. Acad. Sci. 88:11120-3). Such single chain antibodies may also be used for production of anti-idiotypic antibodies for use in this invention.

Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86: 3833-3837; Winter, G. et al. (1991) Nature 349:293-299).

Antibody fragments which contain specific binding sites specific for phage peptides or for use as anti-phage antibodies may also be generated. For example, such fragments include, but are not limited to, the F(ab')2 fragments which can be produced by pepsin digestion of the antibody molecule and the Fab fragments which can be generated by reducing the disulfide bridges of the F(ab')2 fragments. Alternatively, Fab expression libraries may be constructed to allow rapid and easy identification of monoclonal Fab fragments with the desired specificity (Huse, W. D. et al. (1989) Science 254:1275-1281). Such fragments when specific for anti-phage antibodies may be used for production of anti-idiotypic antibodies or fragments thereof.

Monoclonal antibodies of this invention may be “chimeric”, an example of which is an animal antigen-binding variable domain coupled to a human constant domain (Cabilly et al., U.S. Pat. No. 4,816,567; Morrison, S. L. et al., Proc. Natl. Acad. Sci. USA 81:6851-6855 (1984); Boulianne, G. L. et al., Nature 312:643-646 (1984); Neuberger, M. S. et al., Nature 314:268-270 (1985)). The term “chimeric” antibody describes a polypeptide comprising at least the antigen binding portion of an antibody molecule linked to at least part of another protein such as an immunoglobulin constant domain. However, antibodies of this invention may be conjugated to a variety of moieties including labelling moieties.

Various immunoassays may be used for screening to identify antibodies having a desired specificity. Numerous protocols for competitive binding or immunoradiometric assays using either polyclonal or monoclonal antibodies with established specificities are well known in the art. Such immunoassays typically involve the measurement of complex formation between a phage antigen and its specific antibody. Monoclonal-based immunoassays utilizing monoclonal antibodies reactive to at least two non-interfering epitopes are preferred, but competitive binding assays may also be employed (Maddox, D. E. et al. (1983; J. Exp. Med. 158:1211-1216).

This invention involves use of antibodies and phage peptides for diagnostic purposes, in particular for assays to detect or quantify the presence of anti-phage ΦCpn1 antibodies or a phage antigen (e.g. a phage ΦCpn1 peptide) in a sample. Examples of such assays include ELISA (enzyme-linked immunosorbent assays) and “Western blots” which can be used to detect phage ΦCpn1 peptides in samples. Numerous other immunoassays are known in the art (Methods in Cell Biology, Vol. 37: Antibodies in Cell Biology, Asai, ed., Academic Press, Inc., New York (1993); and Basic and Clinical Immunology, 7^{th} ed., Stites & Terr, eds., (1991)).

One methodology for testing for the presence of phage ΦCpn1 or host C. pneumoniae containing the phage in a biological sample according to this invention is to assay for the presence of antibodies to the phage already present in the sample which were produced by the immune system of a patient. In this embodiment, it is preferable to employ one or more ΦCpn1 peptides as a binding moiety for the detection of the antibodies. Preferably, the peptides so employed will be substantially identical to a native ΦCpn1 protein or immunogenic fragment thereof. Preferably, such peptides will be immobilized and/or labelled to facilitate performance of standard immunoassay techniques. In this context, the term “labelled” includes joining of a detectable moiety may be an antibody, an enzyme or a radiolabel. The methodology employed in the Examples below with Vp1 protein may be employed. However, a variety of immunoassays are known in the art whereby binding to antibodies of this invention or antibodies present in a biological sample may be detected.

A preferred method for detecting phage ΦCpn1 proteins is ELISA, in which an antibody is bound to an enzyme (such as peroxidase or phosphatase) which can produce coloured reaction products from an appropriate buffer. ELISA typically utilizes an enzyme tagged antibody to determine an unlabelled antigen of unknown quantity. For example, a phage ΦCpn1 peptide may be coupled to or immobilized in a substrate and is exposed to a sample to be tested for the presence of anti-phage antibodies (first antibody). After washing the substrate is exposed to a labelled antibody capable of binding to the first antibody and the label is detected (e.g. by reaction of an enzyme acting as the label). Detection of the label is indicative of the presence of the first antibody. The sample may be of biological tissue or fluid, such as blood, and may be pretreated as necessary by dilution in a suitable buffer solution (e.g. Tris at physiological pH) or concentrated for use in ELISA. As product of reactions used in ELISA procedures may be coloured, the amount of product formed may readily be determined by the intensity of the colour that has developed using a spectrophotometer. The activity of the bound enzyme is proportional to the amount of the first antibody in the sample.

Another example of a method for detecting phage ΦCpn1 peptides is the Western blot, which makes use of ΦCpn1 specific antibodies such as are described herein. Biologic samples containing proteins can be assayed by fractionation on polyacrylamide gels under denaturing conditions. Alternatively, tris tricine polyacrylamide gel electrophoresis can be used for improved separation of small peptides in the range from 1 to 100 kDa (Schägger H. and von Jagow G. ((1987). Analytical Biochemistry 166: 368-379 and Klafki H.-W. et al. (1996) Analytical Biochemistry 237, 24-29.). The proteins separated in the gels can then be transferred to a membrane using a variety of methods known in the art. Membranes can then be probed using phage ΦCpn1 specific antibodies in a western blot to identify the proteins of interest in the biological sample preparations.

Numerous Western blotting methods are known in the art (ECL western blotting protocol —Amersham; Hsu S M. Methods Enzymol (1990) 184:357-63; Leong M M. and Fox G R. Methods Enzymol (1990) 184:442-51).

Methods of this invention for detecting phage may also include detection of nucleic acids associated with the phage. This methodology may involve amplification of DNA derived from a biological sample in which the amplification is directed towards phage specific nucleic acid sequences. The presence of such amplified sequences may be detected by various means including the use of labelled nucleotide probes. An example of the application of this technology with respect to Chlamydia is described in numbered reference [19]. Such methods are readily performed with knowledge of the phage genome as set out in the Tables which follow.

Tables 1-14 set out the entire phage ΦCpn1 genome (Table 1); the coding regions for the phage proteins (Tables 2-8); and, the predicted amino acids sequences of the phage proteins (Tables 9-14). This sequence information may be found under GenBank accession numbers AE002163 and AE002161.

TABLE 1

| | |
|---|---|
| ΦDCpn1 of Chlamydia pneumoniae AR39 (1-4532) | |
| GATCCTTCGAAAAATCAATTTGCCGCGCATCCTGAAGATTACATTCTCTATGAGATTGGA | (SEQ ID NO: 1) |
| TCTTACGATGACTCTACTGGAACTTTCATTCCCTTAGATGTGCCTAAAGCCTTAGGAACA | |
| GGCTTGGATTTTAAGCACAAACAGTAGGGAAGATATGGTTAGGAATCGGCGTTTGCCTTC | |
| AGTTATGAGTCATTCTTTCGCGCAAGTTCCATCAGCGCGAATTCAGAGAAGTTCTTTTGA | |
| TAGATCTTGTGGTTTAAAGACTACATTCGACGCCGGTTACCTAATCCCTATCTTTTGCGA | |
| TGAAGTTCTCCCTGGAGATACTTTCTCCTTGAAAGAGGCGTTTTTAGCACGTATGGCTAC | |
| GCCTATCTTTCCTCTTATGGATAATTTGCGTTTAGATACGCAGTATTTCTTTGTTCCTCT | |
| TCGACTTATATGGTCGAATTTCCAAAAGTTCTGTGGAGAACAAGATAATCCTGGAGATTC | |
| CACAGATTTTCTTACCCCAGTTTTAACCGCTCCTAGTGGGGGATTTACTGAAGGATCAAT | |
| CCATGATTATCTTGGTCTACCTACTAAAGTTGCAGGAATTGAATGTGTTGCGTTCTGGCA | |
| CAGAGCTTACAATTTGATTTGGAACCAGTACTATCGTGATGAGAATATTCAGGAATCTGT | |
| TGACGTGGAAATGGGAGACACCACCTCTAATGAGGTGAATAATTATAAGCTTCTTAAGCG | |
| TGGGAAGCGTTATGATTACTTCACTTCATGTCTCCCTTGGCCACAAAAAGGTCCTGCAGT | |
| GACAATCGGAGTTGGAGGTATTGTTCCTGTTCAAGGTTTAGGAATTCAATGGGGGAATTC | |
| TAGTGCTCCAAATCCTATAACTGCTTCTAGTTGGATAAATTCTGTTAATCCTACATTTAT | |
| AAATTCTACAACGCCGACGCCTACAGGAACGAATCAGATTTTGAATTATGGTCAGGCGTA | |
| TTATATTAAGAAGCCTGGAGAAGCAACTACAGATCCTACACCTAGGGCTTATGTAGATTT | |
| AGGTTCTACTTCTCCTGTGACGATTAATTCTCTTCGTGAAGCTTTCCAATTGCAAAAGCT | |
| TTATGAGAGAGATGCTCGTGGCGGAACAAGGTACATTGAGATTATTCGTTCCCATTTCAA | |
| TGTGCAGTCTCCAGATGCAAGGTTGCAACGTGCAGAGTATCTTGGAGGTTCTTCAACTCC | |
| TGTGAATATTTCTCCGATTCCACAGACTTCCTCAACAGACTCCACATCTCCTCAAGGAAA | |
| TCTTGCTGCTTATGGTACAGCGATTGGATCGAAGCGAGTCTTCACAAAGTCTTTCACAGA | |
| ACATGGTGTAATTCTTGGATTAGCCTCTGTACGCGCCGATCTCAACTATCAGCAAGGTTT | |
| GGATAGGATGTGGTCACGAAGAACGCGCTGGGACTTTTACTGGCCTGCTCTTAGCCATTT | |
| AGGTGAGCAAGCTGTGCTCAATAAAGAGATCTATTGCCAAGGTCCTGCAGTTAAGGATGC | |
| TCAGAATGGTAATGTTGTTGTGGATGAGCAAGTCTTTGGATATCAGGAGAGATTTGCGGA | |
| GTATCGCTATAAGACTTCGAAAATCACTGGGAAGTTCCGATCAAATGCTACAGGTTCTTT | |
| AGATGCATGGCATTTAGCTCAGCAGTTTGAGAATCTTCCAACACTTTCTCCAGAGTTTAT | |
| CGAAGAAAATCCTCCTATGGATCGTGTTGTTGCTGTAGATACTGAGCCAGATTTTCTCTT | |
| AGATGGTTGGTTTTCATTGCGTTGTGCAAGACCAATGCCTGTCTATTCTGTTCCAGGTCT | |
| TATTGATCATTTCTAATTTCCACTCAGTTTTCCGGTTTGATAAAGCAAACTCACGTTCGT | |
| AGATAAGTGAGTACGGTGAAAACCAAAACGGAAAGCTGAGGCGTAAAAATGTGGAGAATT | |
| TATGAATCCCGAACAACTCACGAGTACTCTCGGTTCAGCAGTTTCTGGAGTTGCCCAAGG | |
| ATTATCCTTCCTCCCTGGAATAGCTTCCGGAGTTTTAGGATATCTTGGCGCACAGAAACA | |
| AAATGCGACTGCGAAGCAAATTGCTAGAGAGCAAATGGCTTTTCAGGAGCGCATGTCTAA | |
| CACGGCATACCAACGTGCTATGGAAGACATGAAGAAAGCTGGCCTTAACCCTATGTTAGC | |
| TTTTTCTAAAGGCGGTGCTTCTTCTCCTGCAGGAGCGTCATGGTCTCCGAATAATCCTGT | |
| AGAGAGCGCGATGAATTCTGGACTTGCCGTGCAAAGACTTACTTATGAACGTAAGAAAAT | |
| GCAGGCAGAGCTTCAGAATCTTCGTGAGCAGAACCGTTTGATTAGAAACCAAGCAATACG | |
| TGAAGGCTATCTCGCAGAACGAGATAAATATATGCGTGTTGCTGGAGTTCCTGTGGCTAC | |
| TGAGATGTTAGATAGAACTTCAGGTCTTCTCTCATCTTCAGCTAAGGCATTTAAGAATCT | |
| TTTTTCAAGAAAAGGAAGGTAGATGTTTAAGTCGGCATATTCCGAAAAAAAATCTGTAAA | |
| GATGAAGTTTACACAGAAATCTTTGACGCAACAACACAACAAAGATGAGTGTGATATTAA | |
| CAACATTGTCGCAAAACTCAACGCTACAGGCGTTTTAGAGCACGTAGAGCGACGATCTCC | |
| ACGTTATATGGACTGTATGGACCCTATGGAGTATTCCGAGGCTCTAAACGTCGTTATTGA | |
| GGCTCAGGAGCAATTTGACTCTTTACCAGCGAAAGTTCGTGAACGTTTTGGAAATGATCC | |
| GGAAGCGATGCTCGATTTCTTGAGCCGTGAAGAAAATTATGAAGAAGCAAAGGCGTTAGG | |
| TTTTGTTTATGAAGATGGAACTTCTGGAGCACCTCAAACATTTTTTGAAGCTGATCCTAA | |
| AGATGATCAAAATGTGGCAAACCAAGAACCTGGATTAGCCCAAAAATGAGCAAATTTTGT | |
| GCAAAAAAGTGTGCAAAAAATGCCCAAAAAAAGGGCCAAAAAATGCCCCCAAAATCGGAG | |
| CATTTTACGAGAGAAAAACACCTAGACTTAAACAGTCTACTTGATCTGTTTAAGTCTAGG | |
| TGATACCGATTCAAGGAAATATTTGAAAAAATAAGCCCTATTTAGGGCCCAAAATTTAAG | |
| CTTAAAATGAGGTTAAAAATGGCACGAAGAAGATACAGACTTCCGCGACGTAGAAGTCGA | |
| AGACTTTTTTCAAGAACTGCATTAAGGATGCATCCAAGAAATAGGCTTCGAAGAATTATG | |
| CGTGGCGGCATTAGGTTCTAGTTTTGGACGTTAAGGAAATCTTTAAGGTTATGCTAAATT | |
| AACTGTCATGTATAATTTGGCTCGTGACCGAATAGTCATATTCGCACTGAATAAATTGTA | |
| CACAACAGTTGAAGGCTTGGATGTTGATTTTTAATGTCTTAGCCTTCATTTTTGGTTTGT | |
| TGTAACTTTAATTTAATTCAGGCATTTATGGCGTGTGTCTCTCCTTTCGTATGTTTTATA | |
| GATTCTTATAACCAGCTCTGGTTTCCCAAAGGTAAGAAGTCTCCTAAACCTTGGGATAAA | |
| GTCCGTGAATTAAATTCTTTTGAGCAAACTCAACCTGAAGAGTATCGAAAACGATGGGTT | |
| GTGATGCCTTGTCTTAAGTGTAGGTTTTGCAGAGTGCGGAATGCAAAGATTTGGTCGTAT | |
| CGTTGCATGCACGAAGCGTCTTTATATTCTCAGAATTGTTTTTTAACTTTGACTTATGAG | |
| GATCGTTATCTTCCAGAGAATGGCTCTTTGGTGAGAAATCATCCTCGTTTGTTTCTTATG | |
| CGATTGAGGAAAGAGATCTATCCTCATAAAATTCGTTATTTTGGATGTGGTGAATATGGA | |
| TCGAAATTACAAAGGCCTCATTATCATCTTCTTATTTATAATTACGATTTTCCTGATAAG | |
| AAGCTCTTGAGTAAAAAGCGTGGCAATCCTCTCTTTGTTTCTGAGAAGTTAATGCGGCTT | |
| TGGCCTTTTGGATTCTCTACAGTAGGATCTGTAACGCGGCAGAGTGCAGGTTATGTAGCG | |
| CGATATTCTTTGAAGAAAGTGAATGGAGATATTTCTCAAGATCATTACGGTCAAAGACTT | |
| CCGGAGTTTCTTATGTGTTCTCTTAAACCAGGAATAGGAGCGGATTGGTATGAGAAATAT | |
| AAACGCGATGTTTATCCTCAGGATTATCTTGTTGTGCAAGATAAAGGGAAGTCTTTTAAG | |
| ACGCGTCCTCCACGTTACTATGATAAGTTACATTCTCGGTTTGATCCGGAAGAGATGGAA | |
| GAGATCAAACAAAAACGCGTAGAGAAATTTATGGCTTTGCCTGAGTTAACTCAAGATAAG | |
| GCTGAGGTGAAGCAATATATTTTCAATGACCGTACGAAGAGACTCTTTAGAGACTATGAG | |
| GAGGAGAGTTACTAAACTTTTTTAAAAAATAGGAGCTTTTTTCAATGAAAGTTTTTACAG | |
| TGTTTGATATTAAGACGGAAATTTATCAGCAGCCTTTCTTTATGCAGGCTACGGGAGCGG | |
| CAATTAGAGCGTTTTCCGATATGGTAAATGAG | |

TABLE 2
All of the predicted coding regions found between
nt 1 and 4532 of C. pneumoniae genome (A39).

| Accession # | Common Name | 5′ End | 3′ End |
|---|---|---|---|
| CPA0001 | Vp1 protein | 155 | 1813 |
| CPA0002 | capsid protein Vp2 | 1922 | 2479 |
| CPA0004 | capsid protein Vp3 | 2483 | 2926 |
| CPA0005 | protein | 2937 | 3041 |
| CPA0006 | protein | 3217 | 3125 |
| CPA0007 | conserved protein | 3412 | 4392 |

TABLE 3

| | |
|---|---|
| >CPA0001 | |
| ATGGTTAGGAATCGGCGTTTGCCTTCAGTTATGAGTCATTCTTTCGCGCAAGTTCCATCA | (SEQ ID NO: 2) |
| GCGCGAATTCAGAGAAGTTCTTTTGATAGATCTTGTGGTTTAAAGACTACATTCGACGCC | |
| GGTTACCTAATCCCTATCTTTTGCGATGAAGTTCTCCCTGGAGATACTTTCTCCTTGAAA | |
| GAGGCGTTTTTAGCACGTATGGCTACGCCTATCTTTCCTCTTATGGATAATTTGCGTTTA | |
| GATACGCAGTATTTCTTTGTTCCTCTTCGACTTATATGGTCGAATTTCCAAAAGTTCTGT | |
| GGAGAACAAGATAATCCTGGAGATTCCACAGATTTTCTTACCCCAGTTTTAACCGCTCCT | |
| AGTGGGGGATTTACTGAAGGATCAATCCATGATTATCTTGGTCTACCTACTAAAGTTGCA | |
| GGAATTGAATGTGTTGCGTTCTGGCACAGAGCTTACAATTTGATTTGGAACCAGTACTAT | |
| CGTGATGAGAATATTCAGGAATCTGTTGACGTGGAAATGGGAGACACCACCTCTAATGAG | |
| GTGAATAATTATAAGCTTCTTAAGCGTGGGAAGCGTTATGATTACTTCACTTCATGTCTC | |
| CCTTGGCCACAAAAAGGTCCTGCAGTGACAATCGGAGTTGGAGGTATTGTTCCTGTTCAA | |
| GGTTTAGGAATTCAATGGGGGAATTCTAGTGCTCCAAATCCTATAACTGCTTCTAGTTGG | |
| ATAAATTCTGTTAATCCTACATTTATAAATTCTACAACGCCGACGCCTACAGGAACGAAT | |
| CAGATTTTGAATTATGGTCAGGCGTATTATATTAAGAAGCCTGGAGAAGCAACTACAGAT | |
| CCTACACCTAGGGCTTATGTAGATTTAGGTTCTACTTCTCCTGTGACGATTAATTCTCTT | |
| CGTGAAGCTTTCCAATTGCAAAAGCTTTATGAGAGAGATGCTCGTGGCGGAACAAGGTAC | |
| ATTGAGATTATTCGTTCCCATTTCAATGTGCAGTCTCCAGATGCAAGGTTGCAACGTGCA | |
| GAGTATCTTGGAGGTTCTTCAACTCCTGTGAATATTTCTCCGATTCCACAGACTTCCTCA | |
| ACAGACTCCACATCTCCTCAAGGAAATCTTGCTGCTTATGGTACAGCGATTGGATCGAAG | |
| CGAGTCTTCACAAAGTCTTTCACAGAACATGGTGTAATTCTTGGATTAGCCTCTGTACGC | |
| GCCGATCTCAACTATCAGCAAGGTTTGGATAGGATGTGGTCACGAAGAACGCGCTGGGAC | |
| TTTTACTGGCCTGCTCTTAGCCATTTAGGTGAGCAAGCTGTGCTCAATAAAGAGATCTAT | |
| TGCCAAGGTCCTGCAGTTAAGGATGCTCAGAATGGTAATGTTGTTGTGGATGAGCAAGTC | |
| TTTGGATATCAGGAGAGATTTGCGGAGTATCGCTATAAGACTTCGAAAATCACTGGGAAG | |
| TTCCGATCAAATGCTACAGGTTCTTTAGATGCATGGCATTTAGCTCAGCAGTTTGAGAAT | |
| CTTCCAACACTTTCTCCAGAGTTTATCGAAGAAAATCCTCCTATGGATCGTGTTGTTGCT | |
| GTAGATACTGAGCCAGATTTTCTCTTAGATGGTTGGTTTTCATTGCGTTGTGCAAGACCA | |
| ATGCCTGTCTATTCTGTTCCAGGTCTTATTGATCATTTC | |

TABLE 4

| | |
|---|---|
| >CPA0002 | |
| ATGAATCCCGAACAACTCACGAGTACTCTCGGTTCAGCAGTTTCTGGAGTTGCCCAAGGA | (SEQ ID NO: 3) |
| TTATCCTTCCTCCCTGGAATAGCTTCCGGAGTTTTAGGATATCTTGGCGCACAGAAACAA | |
| AATGCGACTGCGAAGCAAATTGCTAGAGAGCAAATGGCTTTTCAGGAGCGCATGTCTAAC | |
| ACGGCATACCAACGTGCTATGGAAGACATGAAGAAAGCTGGCCTTAACCCTATGTTAGCT | |
| TTTTCTAAAGGCGGTGCTTCTTCTCCTGCAGGAGCGTCATGGTCTCCGAATAATCCTGTA | |
| GAGAGCGCGATGAATTCTGGACTTGCCGTGCAAAGACTTACTTATGAACGTAAGAAAATG | |
| CAGGCAGAGCTTCAGAATCTTCGTGAGCAGAACCGTTTGATTAGAAACCAAGCAATACGT | |
| GAAGGCTATCTCGCAGAACGAGATAAATATATGCGTGTTGCTGGAGTTCCTGTGGCTACT | |
| GAGATGTTAGATAGAACTTCAGGTCTTCTCTCATCTTCAGCTAAGGCATTTAAGAATCTT | |
| TTTTCAAGAAAAGGAAGG | |

TABLE 5

| | |
|---|---|
| >CPA0004 | |
| ATGTTTAAGTCGGCATATTCCGAAAAAAAATCTGTAAAGATGAAGTTTACACAGAAATCT | (SEQ ID NO: 4) |
| TTGACGCAACAACACAACAAAGATGAGTGTGATATTAACAACATTGTCGCAAAACTCAAC | |
| GCTACAGGCGTTTTAGAGCACGTAGAGCGACGATCTCCACGTTATATGGACTGTATGGAC | |
| CCTATGGAGTATTCCGAGGCTCTAAACGTCGTTATTGAGGCTCAGGAGCAATTTGACTCT | |
| TTACCAGCGAAAGTTCGTGAACGTTTTGGAAATGATCCGGAAGCGATGCTCGATTTCTTG | |
| AGCCGTGAAGAAAATTATGAAGAAGCAAAGGCGTTAGGTTTTGTTTATGAAGATGGAACT | |
| TCTGGAGCACCTCAAACATTTTTTGAAGCTGATCCTAAAGATGATCAAAATGTGGCAAAC | |
| CAAGAACCTGGATTAGCCCAAAAA | |

TABLE 6

| | |
|---|---|
| >CPA0005 | |
| TTGTGCAAAAAAGTGTGCAAAAAATGCCCAAAAAAAGGGCCAAAAAATGCCCCCAAAATC | (SEQ ID NO: 5) |
| GGAGCATTTTACGAGAGAAAAACACCTAGACTTAAACAGTCTACT | |

TABLE 7

| | |
|---|---|
| >CPA0006 | |
| TTGGATGCATCCTTAATGCAGTTCTTGAAAAAAGTCTTCGACTTCTACGTCGCGGAAGTC | (SEQ ID NO: 6) |
| TGTATCTTCTTCGTGCCATTTTTAACCTCATTT | |

TABLE 8

| | |
|---|---|
| >CPA0007 | |
| TTGGTTTGTTGTAACTTTAATTTAATTCAGGCATTTATGGCGTGTGTCTCTCCTTTCGTA | (SEQ ID NO: 7) |
| TGTTTTATAGATTCTTATAACCAGCTCTGGTTTCCCAAAGGTAAGAAGTCTCCTAAACCT | |
| TGGGATAAAGTCCGTGAATTAAATTCTTTTGAGCAAACTCAACCTGAAGAGTATCGAAAA | |
| CGATGGGTTGTGATGCCTTGTCTTAAGTGTAGGTTTTGCAGAGTGCGGAATGCAAAGATT | |
| TGGTCGTATCGTTGCATGCACGAAGCGTCTTTATATTCTCAGAATTGTTTTTTAACTTTG | |
| ACTTATGAGGATCGTTATCTTCCAGAGAATGGCTCTTTGGTGAGAAATCATCCTCGTTTG | |
| TTTCTTATGCGATTGAGGAAAGAGATCTATCCTCATAAAATTCGTTATTTTGGATGTGGT | |
| GAATATGGATCGAAATTACAAAGGCCTCATTATCATCTTCTTATTTATAATTACGATTTT | |
| CCTGATAAGAAGCTCTTGAGTAAAAAGCGTGGCAATCCTCTCTTTGTTTCTGAGAAGTTA | |
| ATGCGGCTTTGGCCTTTTGGATTCTCTACAGTAGGATCTGTAACGCGGCAGAGTGCAGGT | |
| TATGTAGCGCGATATTCTTTGAAGAAAGTGAATGGAGATATTTCTCAAGATCATTACGGT | |
| CAAAGACTTCCGGAGTTTCTTATGTGTTCTCTTAAACCAGGAATAGGAGCGGATTGGTAT | |
| GAGAAATATAAACGCGATGTTTATCCTCAGGATTATCTTGTTGTGCAAGATAAAGGGAAG | |
| TCTTTTAAGACGCGTCCTCCACGTTACTATGATAAGTTACATTCTCGGTTTGATCCGGAA | |
| GAGATGGAAGAGATCAAACAAAAACGCGTAGAGAAATTTATGGCTTTGCCTGAGTTAACT | |
| CAAGATAAGGCTGAGGTGAAGCAATATATTTTCAATGACCGTACGAAGAGACTCTTTAGA | |
| GACTATGAGGAGGAGAGTTAC | |

TABLE 9

| | |
|---|---|
| >CPA0001 | |
| MVRNRRLPSVMSHSFAQVPSARIQRSSFDRSCGLKTTFDAGYLIPIFCDEVLPGDTFSLK | (SEQ ID NO: 8) |
| EAFLARMATPIFPLMDNLRLDTQYFFVPLRLIWSNFQKFCGEQDNPGDSTDFLTPVLTAP | |
| SGGFTEGSIHDYLGLPTKVAGIECVAFWHRAYNLIWNQYYRDENIQESVDVEMGDTTSNE | |
| VNNYKLLKRGKRYDYFTSCLPWPQKGPAVTIGVGGIVPVQGLGIQWGNSSAPNPITASSW | |
| INSVNPTFINSTTPTPTGTNQILNYGQAYYIKKPGEATTDPTPRAYVDLGSTSPVTINSL | |
| REAFQLQKLYERDARGGTRYIEIIRSHFNVQSPDARLQRAEYLGGSSTPVNISPIPQTSS | |
| TDSTSPQGNLAAYGTAIGSKRVFTKSFTEHGVILGLASVRADLNYQQGLDRMWSRRTRWD | |
| FYWPALSHLGEQAVLNKEIYCQGPAVKDAQNGNVVVDEQVFGYQERFAEYRYKTSKITGK | |
| FRSNATGSLDAWHLAQQFENLPTLSPEFIEENPPMDRVVAVDTEPDFLLDGWFSLRCARP | |
| MPVYSVPGLIDHF | |

TABLE 10

| | |
|---|---|
| >CPA0002 | |
| MNPEQLTSTLGSAVSGVAQGLSFLPGIASGVLGYLGAQKQNATAKQIAREQMAFQERMSN | (SEQ ID NO: 9) |
| TAYQRAMEDMKKAGLNPMLAFSKGGASSPAGASWSPNNPVESAMNSGLAVQRLTYERKKM | |
| QAELQNLREQNRLIRNQAIREGYLAERDKYMRVAGVPVATEMLDRTSGLLSSSAKAFKNL | |
| FSRKGR | |

TABLE 11

| | |
|---|---|
| >CPA0004 | |
| MFKSAYSEKKSVKMKFTQKSLTQQHNKDECDINNIVAKLNATGVLEHVERRSPRYMDCMD | (SEQ ID NO: 10) |
| PMEYSEALNVVIEAQEQFDSLPAKVRERFGNDPEAMLDFLSREENYEEAKALGFVYEDGT | |
| SGAPQTFFEADPKDDQNVANQEPGLAQK | |

TABLE 12

(SEQ ID NO: 11)

>CPA0005
MCKKVCKKCPKKGPKNAPKIGAFYERKTPRLKQST

TABLE 13

| | |
|---|---|
| >CPA0006 | |
| MDASLMQFLKKVFDFYVAEVCIFFVPFLTSF | (SEQ ID NO: 12) |

TABLE 14

| | |
|---|---|
| >CPA0007 | |
| MVCCNFNLIQAFMACVSPFVCFIDSYNQLWFPKGKKSPKPWDKVRELNSFEQTQPEEYRK | (SEQ ID NO: 13) |
| RWVVMPCLKCRFCRVRNAKIWSYRCMHEASLYSQNCFLTLTYEDRYLPENGSLVRNHPRL | |
| FLMRLRKEIYPHKIRYFGCGEYGSKLQRPHYHLLIYNYDFPDKKLLSKKRGNPLFVSEKL | |
| MRLWPFGFSTVGSVTRQSAGYVARYSLKKVNGDISQDHYGQRLPEFLMCSLKPGIGADWY | |
| EKYKRDVYPQDYLVVQDKGKSFKTRPPRYYDKLHSRFDPEEMEEIKQKRVEKFMALPELT | |
| QDKAEVKQYIFNDRTKRLFRDYEEESY | |

Nucleic acids and peptides for use in this invention may correspond in sequence to peptides or nucleic acids of phage ΦCpn1. Preferably, such corresponding peptides will comprise at least about 5, more preferably at least about 10, and even more preferably at least about 15 amino acids of a phage ΦCpn1 protein. Preferably, nucleic acids of this invention will comprise at least about 15, more preferably at least about 30 and even more preferably at least about 45 nucleotides corresponding to phage DNA encoding a phage protein.

One measure of “correspondence” of nucleic acids or peptides for use in this invention with reference to the above described phage nucleic acids and peptides is relative “identity” between sequences. In the case of peptides, or in the case of nucleic acids defined according to a encoded peptide correspondence includes a peptide having at least about 50% identity, more preferably at least about 90% identity, even more preferably about 95% and most preferably at least about 98-99% identity to a specified peptide. Preferred measures of identity as between nucleic acids is the same as specified above for peptides with at least about 90% or at least about 98-99% identity being most preferred.

The term “identity” as used herein refers to the measure of the identity of sequence between two peptides or between two nucleic acid molecules. Identity can be determined by comparing a position in each sequence which may be a line for purposes of comparison. Two amino acid or nucleic acid sequences are considered substantially identical if they share at least about 75% sequence identity, preferably at least about 90% sequence identity and even more preferably at least 95% sequence identity and most preferably at least about 98-99% identity.

Sequence identity may be determined by the BLAST algorithm currently is use and which was originally described in Altschul et al. (1990) J. Mol. Biol. 215:403-410. The BLAST algorithm may be used with the published default settings. When a position in the compared sequence is occupied by the same base or amino acid, the molecules are considered to have shared identity at that position. The degree of identity between sequences is a function of the number of matching positions shared by the sequences.

An alternate measure of identity of nucleic acid sequences is to determine whether two sequences hybridize to each other under low stringency, and preferably high stringency conditions. Such sequences are substantially identical when they will hybridize under high stringency conditions. Hybridization to filter-bound sequences under low stringency conditions may, for example, be performed in 0.5 M NaHPO₄, 7% sodium dodecyl sulfate (SDS), 1 mM EDTA at 65° C., and washing in 0.2×SSC/0.1 SDS at 42° C. (see Ausubel et al. (eds.) 1989, Current Protocols in Molecular Biology, Vol. 1, Green Publishing Associates, Inc., and John Wiley & sons, Inc., New York, at p. 2.10.3). Alternatively, hybridization to filter-bound sequences under high stringency conditions, may for example, be performed in 0.5 M NaHPO₄, 7% (SDS), 1 mM EDTA at 65° C., and washing in 0.2×SSC/0.1% SDS at 68° C. (see Ausubel et al (eds.) 1989, supra). Hybridization conditions may be modified in accordance with known methods depending on the sequence of interest (see Tijssen, 1993, Laboratory Techniques in Biochemistry and Molecular Biology —Hybridization with Nucleic Acid Probes, Part I, Chapter 2 “Overview of Principles in Hybridization and the Strategy of Nucleic Acid Probe Assays”, Elsevier, New Yrok). Generally, stringent conditions are selected to be about 5° C. lower than the thermal melting point for the specific sequence at a defined ionic strength and pH.

The method of this invention may comprise the collection of a biological sample. Many methods are known in the art for collecting bodily samples and extracting genetic material from those samples. Genetic material can be extracted from blood, tissue and hair and other bodily samples. There are many known methods for the separate isolation of DNA and RNA from biological material. Typically, DNA is isolated from a biological sample when first the sample is lysed and then the DNA is isolated from the lysate according to any one of a variety of multi-step protocols, which can take varying lengths of time. The methods described in the Examples below may be employed.

Frequently recommended DNA isolation methods involve the use of toxic phenol (Sambrook, J. et al., “Molecular Cloning”, Vol. 2, pp. 9.14-9.23, Cold Spring Harbor Laboratory Press (1989) and Ausubel, Frederick M. et al., “Current. Protocols in Molecular Biology”, Vol. 1, pp. 2.2.1-2.4.5, John Wiley & Sons, Inc. (1994)). Usually, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mixture of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech.), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465.) involves lysing cells in 6M guanidine hydrochloride, precipitating DNA from the lysate at acid pH by adding 2.5 volumes of ethanol, and washing the DNA with ethanol.

Numerous other methods are known in the art, such as the one described by Chomczynski (U.S. Pat. No. 5,945,515), whereby genetic material can be extracted efficiently in as little as twenty minutes. Evans and Hugh (U.S. Pat. No. 5,989,431) describe methods for isolating DNA using a hollow membrane filter.

One method for extracting total RNA is the acid guanidinium thiocyanate-phenol-chloroform method of Chomczynski P. and Sacchi N. (1987) as described in Anal. Biochem. 162:156-159.

Genetic material obtained from a sample may be analysed to determine whether the host C. pneumoniae bacterium or the ΦCpn1 phage is present. Such analysis can be accomplished by any one of a number of possible methods known in the art. A nucleic acid molecule of the invention can be amplified using cDNA, mRNA or genomic DNA as a template and appropriate oligonucleotide primers according to standard PCR amplification techniques or reverse transcription (RT-PCR) techniques such as those described in the Examples below. The amplified nucleic acids can be identified based on their size when visualized using gel electrophoresis. Further confirmation of the identity of amplified nucleic acids can be accomplished by restriction enzyme digestion prior to visualization on a gel, whereby the expected sequence of the amplified PCR product will be cut by a restriction enzyme to produce bands of a predicted size (Gaitanis G. et al. Clin Microbiol Infect (2002) 8 (3): 162-73). Alternatively, the nucleic acid so amplified can be further cloned into an appropriate vector and characterized by DNA sequence analysis.

Oligonucleotides corresponding to all or a portion of a nucleic acid molecule of the invention can be prepared by standard synthetic techniques, e.g., using an automated DNA synthesizer. Synthesis of specific oligonucleotide sequences to be used as primers or hybridization probes can be accomplished using an automated DNA synthesizer such as the Applied Biosystems 380B. Oligonucleotides can be deprotected under standard conditions and purified with ammonia-butanol (Sawadogo M. and van Dyke M. W. (1991) Nucleic Acid Res. 19:674).

An example of a methodology for testing for the presence of phage ΦCpn1 or C. pneumoniae containing the phage in a biological sample taken from a patient is to assay for the presence of nucleic acids using the Polymerase Chain Reaction (PCR) such as is generally described in Innis et al. (1990- PCR Protocols: A guide to methods and applications, Academic Press, Inc.), where primers are designed to selectively amplify only phage ΦCpn1 or C. pneumoniae nucleic acids. By selecting a phage specific sequence primers such as those described below can be designed that will selectively amplify phage DNA in a PCR assay, which can later be visualized using gel electrophoresis. Alternatively, DNA samples can be identified using hybridization techniques as described in U.S. Pat. No. 6,270,961 & 6,025,136. Again probes can be designed that will selectively hybridize to phage DNA, thus allowing for the identification of DNA samples that contain phage ΦCpn1.

DNA sequence analysis can also be performed to determine if phage ΦCpn1 DNA is present in a sample, using one of many sequencing techniques known in the art. One such technique is the Maxam-Gilbert technique (Maxam A M. and Gilbert W. Proc. Natl. Acad. Sci. USA (1977) 74 (4): 560-564) which involves the specific chemical cleavage of terminally labelled DNA. In this technique four samples of the same labelled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labelling.

Another method of DNA sequence analysis is the dideoxy method of sequencing published by Sanger et al. (Sanger et al. Proc. Natl. Acad. Sci. USA (1977) 74 (12): 5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures.

RNA sequencing methods are also known and may be employed in this invention to detect the presence of phage nucleic acids. For example, reverse transcriptase with dideoxy-nucleotides have been used to sequence encephalomyocarditis virus RNA (Zimmern D. and Kaesberg P. Proc. Natl. Acad. Sci. USA (1978) 75 (9): 4257-4261). Mills D R. and Kramer F R. (Proc. Natl. Acad. Sci. USA (1979) 76 (5): 2232-2235) describe the use of Q. beta. replicase and the nucleotide analog inosine for sequencing RNA in a chain-termination mechanism. Direct chemical methods for sequencing RNA are also known (Peattie D A. Proc. Natl. Acad. Sci. USA (1979) 76 (4): 1760-1764). Other methods include those of Donis-Keller et al. (1977, Nucl. Acids Res. 4:2527-2538), Simoncsits A. et al. (Nature (1977) 269 (5631): 833-836), Axelrod V D. et al. (Nucl. Acids Res. (1978) 5 (10): 3549-3563), and Kramer F R. and Mills D R. (Proc. Natl. Acad. Sci. USA (1978) 75 (11): 5334-5338).

Nucleic acid sequences can also be determined by “reading” the sequence through stimulation of natural fluorescence of a cleaved nucleotide with a laser while the cleaved nucleotide is present in a fluorescence enhancing matrix (U.S. Pat. No. 5,674,743).

Methods for preparation of compositions comprising peptides or nucleic acids for the purpose of eliciting an immune response (including for directing expression of the nucleic acid in a subject), are known in the art. Such compositions in accordance with this invention may be used for administration to human or animal subjects.

Methods and medicaments for treatment of Chlamydia infection (including reduction or elimination of infection) are known and may be employed in a subject at risk for vascular damage or shown to be infected with the phage host Chlamydia.

### EXAMPLES

Strains of C. pneumoniae, Growth and Purification

C. pneumoniae strains used in this study are described in Table 15. The human HL cell line was used for growth and propagation of the C. pneumoniae strains. Elementary bodies (EBs) were purified on discontinuous gradients of Renografin-76® (Squibb Canada, Montreal, Canada) as described by Schachter, J., et al. (1994) Methods Enzymol. 236:377-90. Purified EBs were resuspended in isotonic sucrose-phosphate-glutamate buffer and stored at −80° C.

PCR Analysis

PCR analysis was performed using a PTC-200™ Peltier Thermal Cycler (MJ Research). The reaction mixture contained 1.5 mM MgCl₂, 200 μM deoxynucleoside triphosphates, 5 U of Taq DNA polymerase enzyme, and 25 pmol of oligonucleotide primers (for the vpl gene: forward 5′-CGCTCCTAGTGGGGGATTTACTGA-3′ [SEQ ID NO:14]; reverse 5′-CACAGCTTGCTCACCTAAATGGCT-3′ [SEQ ID NO:15]; for the 16s RNA gene: forward 5′-CGGTAATACGGAGGGTGCTAGC-3′ [SEQ ID NO:16]; reverse 5′-GAATTAAACCACATGCTCCACTGC-3′ [SEQ ID NO:17]; for the C. pneumoniae CP0543 gene: forward 5′-CTGTAAGGTGAAAAGTTTTTA-3′ [SEQ ID NO:18]; reverse 5′-CAGCTGTAAATGCAGCTTT-3′ [SEQ ID NO:19]) in a total volume of 50 μl. The PCR cycling conditions were as follows: one cycle of 95° C. for 3 min and 35 cycles of 94° C. for 15 sec, 55° C. for 30 sec, and 72° C. for 2 min. This was followed by strand elongation for 10 min at 72° C.

RT-PCR

Total RNA from HL cells infected with C. pneumoniae strains AR39 or CWL029 was isolated using Trizol™ (Life Technologies) and treated with Rnase-free Dnase (Roche). These RNA samples were used as templates for reverse transcription (RT) in a 20-μl reaction mixture containing 2 μg random hexamers (Roche), 1 μl of 10 mM deoxyribonucleosides triphosphate, 2 μl of 0.1M DTT, 1 μl of RNasin™ (Promega), and 200U Superscript II™ (Life Technologies). RT was carried out at 42° C. for 50 min. PCR was performed with PTC-200 Peltier Thermal Cycler (MJ Research). The reaction mixture contained 1.5 mM MgCl₂, 200 μM deoxynucleoside triphosphates, 5 U of Taq DNA polymerase enzyme, 25 pmol of each oligonucleotide primers specific for vpl gene or 16s RNA gene (same as given above) and 2 μl of RT product in a total volume of 50 μl. The PCR cycling conditions were as follows: one cycle of 95° C. for 3 min and 35 cycles of 94° C. for 15 sec, 55° C. for 30 sec, and 72° C. for 2 min. This was followed by strand elongation for 10 min at 72° C.

Molecular Cloning, Expression and Purification of Recombinant Vp1

vpl DNA fragments were generated by PCR using genomic DNA isolated from C. pneumoniae AR39 as the template. In order to subclone the PCR product as a NcoI and XhoI fragment into pET30b (+) vector (Novagen), forward (5′-AGT AGG GAA GCC ATG GTT AGG-3′; SEQ ID NO:20) and reverse (5′-ACT GAC TCG AGA TTA GAA ATG ATC AAT-3′; SEQ ID NO:21) PCR primers were designed to contain NcoI and XhoI restriction sites, respectively. PCR reactions were carried out using Platinum™ Pfx DNA polymerase (Gibco BRL). The reaction mixture contained 1 mM MgSO₄, 300 μM deoxynucleoside triphosphates, 2.5 U of the Pfx DNA polymerase enzyme, and 25 pmol of each oligonucleotide primer in a total volume of 50 μl. The PCR cycling conditions were as follows: one cycle of 95° C. for 2 min and 35 cycles of 94° C. for 15 sec, 58° C. for 30 sec, and 68° C. for 2 min. This was followed by strand elongation for 10 min at 68° C. The PCR product was purified with the QIAquick™ PCR purification kit (Qiagen) and the purified DNA fragment was cloned into pET30b (+) after restriction enzyme digestion using standard molecular biology techniques. The sequence of the subcloned vpl gene was confirmed by sequencing with dye-labelled terminators using the ABI PRISM™ kit (PE Biosystems). Plasmids carrying the vpl gene (pET-vpl) were transformed into the E. coli strain BL21 (DE3) where Vp1 expression was carried out by inducing the lac promoter for expression of T7 RNA polymerase using isopropyl-β-D-thiogalactoside pyranoside. The expressed Vp1 protein with N-terminal His-tag was purified by nickel column using an His binding purification system (Qiagen).

Antibody Production Against Recombinant Yp1

Antisera against Vp1 was raised in Balb/c mice (Charles River Canada) by intraperitoneal injection of the recombinant proteins (100 μg protein in incomplete Freund adjuvant), followed by two booster injections (50 μg protein in incomplete Freund adjuvant) at two week intervals. Sera were collected and pooled four weeks after the final boost.

Western Blotting

Samples for Western analysis were prepared by boiling purified elementary bodies of C. pneumoniae strains AR39 and CWL 029 for 5 minutes in the protein sample buffer. The samples were subjected to electrophoresis in 10% SDS-PAGE gels according to Laemmli (1970) Nature 227:680-5. The gels were blotted onto nitrocellulose membranes (BioRad) at 70V for 1 h in blotting buffer according to Sambrook et al. (1989) “Molecular Cloning. A Laboratory Manual”, Cold spring Harbor Laboratory (N.Y.)). The filters were blocked overnight with TBS containing 3% BSA at 4° C. prior to incubation with polyclonal anti Vp1 antibodies and peroxidase conjugated sheep anti mouse IgG secondary antibodies. The blots were processed for colour detection using the substrate 5-bromo-4-chloro-3indolyl phosphate/nitro blue tetrazolium.

Assay for ΦCpn1 host Chlamydia Elementary Bodies

C. pneumoniae strain AR39 was grown in HL cells and used as antigen in a microimmunofluorescence test. Formalin-fixed whole elementary bodies of AR-39 were fixed to microscope slides and sera were diluted two-fold beginning at 1:8 to 1:2048. Immunoglobulin G (IgG), IgA and IgM serum antibody fractions were measured using fluorescein isothiocyanate-conjugated heavy chain specific goat anti-mouse IgG, IgA or IgM. The end point titer was recorded as the final dilution giving a discreet fluorescent pattern to the C. pneumoniae elementary bodies.

Selection of Cases and Controls

The seropidemiologic analysis described herein was based on frozen serum specimens (stored at −70° C.) from a subset of participants in a previous case study of risk factors for AAA [20]. The previous study was performed among ambulatory patients attending either of the ultrasound departments of the two tertiary care hospitals in Winnipeg, Manitoba, Canada between June, 1992 and December, 1995. In the previous study, there were 98 AAA cases and 102 non-AAA controls. To minimize selection and misclassification biases, controls were selected from among persons who were undergoing ultrasound examinations for similar indications as the cases. Analysis was restricted to 32 cases and 40 controls from the original study for whom sufficient frozen serum specimens were available.

Two groups of cases and controls were defined in the previous study based on the indication for ultrasound. The first group included persons who had been referred for aortic ultrasound due to clinical suspicion of abdominal aortic aneurysm (AAA). Cases and controls for the second group included persons who underwent abdominal ultrasound for reasons other than suspected AAA. Only persons aged 40 years and older were included. The definition of an AAA was based on the assessment of the ultrasound radiologists at the two ultrasound departments. Specific size criteria were not used. Instead, the definition was based on the shape of the infrarenal aorta such that any definite focal widening was classified as an AAA [21]. Nevertheless, all of the AAA cases and no control had an infrarenal aortic dilatation≧3.0 cm.

Data Collection and Risk Factor Definition

An in-person interview was conducted with each study participant using a standardized questionnaire to collect data related to socio-demographics, symptomatology, indications for ultrasound examination, history of specific medical conditions, family history of AAA and other conditions, and cigarette smoking. Two blood pressure measurements were taken with the patient seated using a standard mercury sphygmomanometer. Fasting serum total-cholesterol levels were measured with the Hitachi-Boehringer-Mannheim 717-Autoanalyzer™ using established protocols.

Participants were classified as having hypertension if they had a systolic blood pressure≧140 mm Hg, a diastolic blood pressure≧90 mm Hg, or they were currently taking anti-hypertensive medication prescribed by a physician. Hypercholesterolemia was defined as the presence of a fasting serum total-cholesterol>6.2 mmol/litre or current use of prescribed lipid-lowering medication. Lifelong pack-years of cigarette smoking exposure were assessed by determining the average packsof cigarettes smoked per day for specific age periods, beginning with the age at initiation. These data were then cumulated to estimate the total pack-years of exposure.

Serological Assays for AR39 Elementary Bodies (EB) and Bacteriophage ΦCpn1 Vp1

Methods for detection of antibodies to C pneumoniae elementary bodies (EB) are known [10; 22; 23]. In this example, detection of both anti-EB and anti-Vp1 antibodies in human immune sera was done using ELISA following the method described in numbered reference [22]. Flat bottomed (96-well), polystyrene, microtiter plates were coated with 100 μl of either C. pneumoniae AR39 EB or recombinant phage Vp1 protein (0.1 μg/well), or coating buffer alone to each well. Plates were incubated overnight at 4° C. and then washed three times with tris buffered saline (TBS). Nonspecific binding sites were blocked by adding 150 μl of 3% BSA in MTPBS (150 mM NaCl, 16 mM Na₂HPO₄, 4 mM NaH₂PO₄, pH 7.3)—Tween 20 detergent buffer to each well for 2 hours at 37° C. After three washings with TBS, 100 μl of human sera diluted to 1:500 were added and incubated for 1 hour at 37° C. After three more washings with TBS, 100 μl of 1:2000 diluted peroxidase conjugated goat anti-human secondary antibody was added and incubated for 1 hour at 37° C. After five washings with TBS, 100 μl of 2,2′-azino-bis(3-ethylbenzthiazoline-6-sulfonic acid) substrate was added and allowed to develop in the dark for five minutes. Readings were made at OD₄₀₅ on a microplate reader.

Statistical Analyses

For all analyses, the presence of antibodies to C. pneumoniae AR39 EB and ΦCpn1 Vp1 in serum was dichotomized such that those with measured levels of ≧0.5 optical density (OD) units were classified as seropositive. Odds ratios, 95% confidence intervals and chi square analyses were used to assess the relationship between these variables and abdominal aortic aneurysm. Unconditional logistic regression was used to compute adjusted odds ratios and 95% confidence intervals for the association between abdominal aortic aneurysm and seropositivity to C. pneumoniae EB and Vp1. Other variables included in the logistic regression model were age, gender, pack-years of smoking, the presence of hypertension and the presence of hypercholesterolemia. Altogether data from 32 AAA cases and 40 controls were analyzed.

Differentiation of C. pneumoniae Strains

Genome sequencing showed that strain AR39 is a phage-containing strain and CWL029 is a phage-free strain [15, 16]. The DNA sequence of the phage was originally found during the genome sequencing of the strain AR39 and no functional studies have been performed so far on this phage. PCR, RT-PCR, and Western blot analysis was performed following the methods described above to demonstrate the determination of the presence and expression of ΦCpn1 vpl gene in one C. pneumoniae strain (AR39) in comparison to another strain (CWL029) that lacks this phage. Results from PCR (FIG. 1A), RT-PCR (FIG. 1B) and Western blot (FIG. 1C) analysis confirm the presence and expression of the phage vpl gene in C. pneumoniae strain AR39 and its absence in strain CWL029.

Distribution of ΦCpn1 among C. pneumoniae Strains

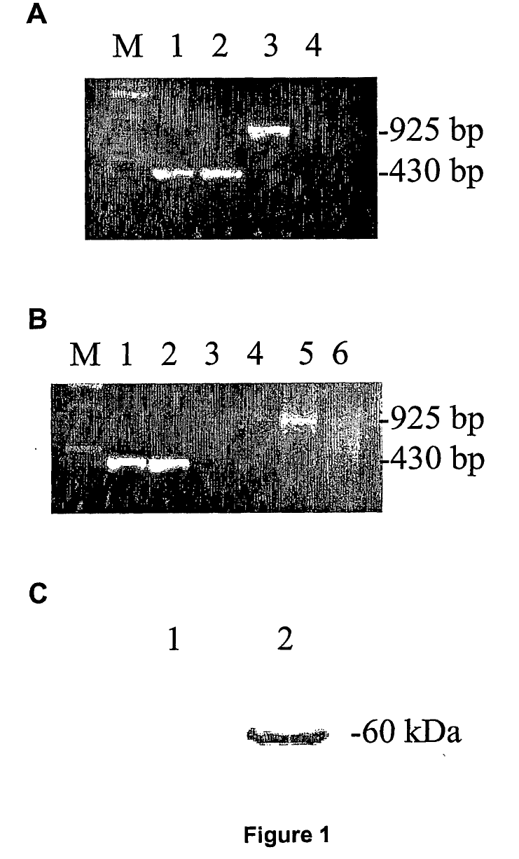

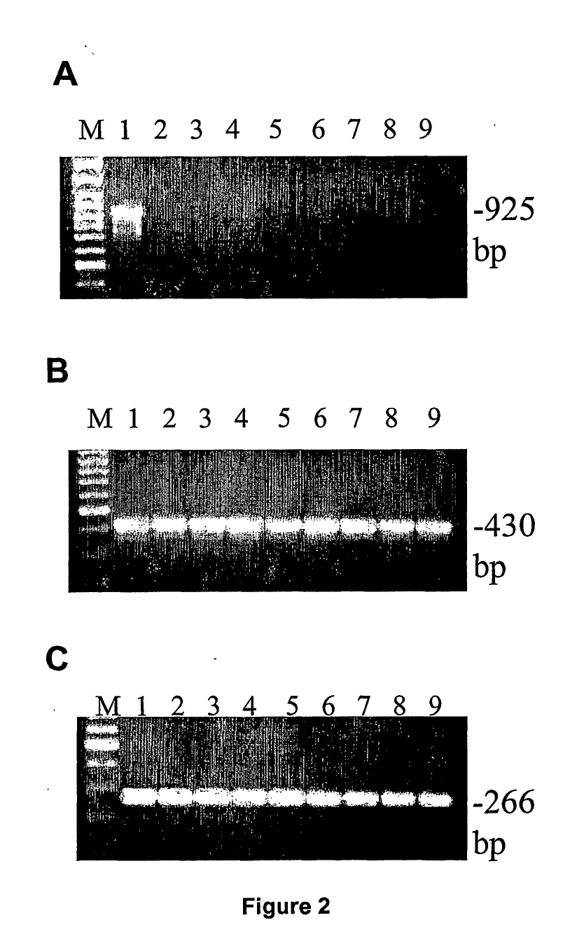

Among the three C. pneumoniae genomes sequenced to date, one (AR39) contains the phage ΦCpn1 [15, 16, 17]. PCR analysis was performed by the method described above to determine the presence of ΦCpn1 vpl gene, in 6 clinical isolates of C. pneumoniae, in addition to the lab strains AR39 and CWL029. The results (FIG. 2) are summarized in Table 15.

TABLE 15
Chlamydia pneumoniae strains evaluated for phage.

| | C. pneumoniae | ΦCpn1- | |
|---|---|---|---|
| | CP0543 | specific | |
| Strain | sequence | sequences | Reference or Source |
| AR39 | Yes | Yes | Grayston [11] |
| CWL029 | Yes | No | Kuo, C., et al. (1988) J. Clin. |
| | | | Microbiol. 26: 812-5. |
| CM1 | Yes | No | Black, C. M., et al. (1991) |
| | | | J. Clin. Microbiol. 29: 1312-6. |
| Kajaani7 | Yes | No | Kajaani, Finland (Dr. P. Saikku) |
| Bay 13 | Yes | No | Spartanburg, South Carolina, |
| | | | USA (Dr. M. Hammerschlag) |
| Bay 16 | Yes | No | Toledo, Ohio, USA |
| | | | (Dr. M. Hammerschlag) |
| Bay 19 | Yes | No | Baltimore, Maryland, USA |
| | | | (Dr. M. Hammerschlag) |
| Bay 20 | Yes | No | Springfield, Missouri, USA |
| | | | (Dr. M. Hammerschlag) |
| Bay 21 | Yes | No | Mesa, Arizona, USA |
| | | | (Dr. M. Hammerschlag) |

ELISA Design and Specificity

In order to demonstrate ΦCpn1 Vp1 protein as an antigen to detect the presence of the phage antibodies in sera collected from controls and cases of abdominal aortic aneurysm (AAA), antibodies are raised in mice using recombinant Vp1 protein generated in Escherichia coli. The Vp1 antigen was highly immunogenic in mice and antibodies thus generated readily recognize the Vp1 in C. pneumoniae EBs (FIG. 1C). Antiserum from non-immunized mouse did not give any bands for C. pneumoniae EBs.

ELISA was used to measure human serum antibodies in 32 AAA cases and 40 controls. There were no significant differences in age, gender distribution, smoking history or history of hypertension between the original study participants (98 cases and 102 controls) and the subset analyzed for this study. Using a cutoff value of 0.5 OD for both C pneumoniae EB and ΦCpn1 Vp1 antibody levels, 61 (84.7%) of the 72 participants were seropositive for C. pneumoniae EB and 42 (58.30%) were seropositive for ΦCpn1 Vp1. Of the 61 who were seropositive for C pneumoniae EB, 39 (63.9%) were also positive for ΦCpn1 Vp1. There were no consistent variations by age, gender, or smoking history for either C. pneumoniae EB or ΦCpn1 Vp1 seropositivity.

A comparison was made between seropositivity for C. pneumoniae IgG, IgA and IgM antibodies, based on microimmunofluorescence testing, and seropositivity for ΦCpn1 Vp1 by ELISA. Of the 64 subjects who were microimmunofluorescent IgG seropositive, 39 (60.9%) were seropositive for Vp1. Of the 36 subjects who were IgA seropositive, 24 (66.7%) were seropositive for Vp1. Of the 2 subjects who were IgM seropositive, one was seropositive for Vp1.

Correlation Between c. pneumoniae EB and ΦCpn1 Vp1 Antibodies and Abdominal Aortic Aneurysm

Table 16 shows the relation between AAA and C. pneumoniae EB and bacteriophage Vp1 seropositivity. Seropositivity (i.e. levels>0.5 OD) was more common among cases than controls for both C. pneumoniae EB (90.6% vs. 80.0%) and ΦCpn1 Vp1 (68.8% vs. 50.0%). There was a positive association with AAA for both C. pneumoniae EB (crude OR 2.4, [95% C.I., 0.6-10.0]) and ΦCpn1 Vp1 (crude OR 2.2, [0.8-5.8]) seropositivity. Multivariate analysis to control for age, gender, pack-years of smoking, hypertension and hypercholesterolemia showed a persistent association between AAA and C. pneumoniae EB seropositivity (adjusted OR 2.3 [0.5-11.5]). Strikingly, multivariate adjustment resulted in a much stronger association between AAA and ΦCpn1 Vp1 seropositivity (adjusted OR 4.2 [1.2-14.4]) (p=0.02). Seropositivity to both C. pneumoniae EB and ΦCpn1 Vp1 (versus seronegativity to both) was even more strongly associated with AAA (adjusted OR, 13.9 [1.1-175]). Furthermore, the strong association between AAA and ΦCpn1 Vp1 seropositivity persisted when adjusted for seropositivity to C. pneumoniae EB (adjusted OR, 3.9 [1.1-13.9]) (p=0.04).

TABLE 16
The association between abdominal aortic aneurysm and
seropositivity to C pneumoniae elementary bodies (EB) and the
ΦCpn1 Vp1, based on a case-control analysis in Winnipeg, Canada.

| | Seropositivity (%) | | | |
|---|---|---|---|---|
| | AAA | | | |
| | Cases | Controls | Crude OR | Adjusted* OR |
| | (n = 32) | (n = 40) | (95% C.I.) | (95% C.I.) |
| C | 90.6 | 80.0 | 2.4 (0.6-10.0) | 2.3 (0.5-11.5) |
| pneumoniae | | | | |
| EB‡ | | | | |
| ΦCpn1 | 68.8 | 50.0 | 2.2 (0.83-5.8) | 4.2 (1.2-14.4) |
| Vp1§ | | | | |
| Both C. | 62.5 | 47.5 | 7.4 (0.83-65.7)† | 13.9 (1.1-175)† |
| pneumoniae | | | | |
| EB and | | | | |
| ΦCpn1 Vp1 | | | | |

*Odds ratios adjusted for age, sex, pack-years of smoking, hypertension and hypercholesterolemia using multiple logistic regression.
†compared to a reference category of seronegativity to both C. pneumoniae EB and bacteriophage Vp1.
‡antibodies to C. pneumoniae AR39 elementary body (EB).
§antibodies to the viral protein (Vp)1 of bacteriophage (ΦCpn1).

Overall, 84.7% of the subjects were seropositive for C. pneumoniae EB, and 63.9% of those were also seropositive for Vp1. There was a positive association between Vp1 seropositivity and AAA (adjusted odds ratio (OR) 4.2; 95% confidence interval (CI) 1.2-14.4). The association between C. pneumoniae EB was weaker (adjusted OR 2.3; 95% CI 0.5-11.5). Compared to those who were seronegative to both, seropositivity to both Vp1 and C. pneumoniae EB was strongly associated with AAA (OR 13.9; 95% CI 1.1-175).

A subset (64.50%) of persons who are seropositive to C. pneumoniae have serological evidence of infection to C. pneumoniae strains containing ΦCpn1. Exposure to this specific strain of C. pneumoniae is better correlated with the presence of abdominal aortic aneurysm than is seropositivity to C. pneumoniae in general. Thus, there is a persistent association between Vp1 seropositivity and AAA among subjects who are seropositive to C. pneumoniae, and a much stronger association when seropositivity to both is compared to seronegativity to both.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the described invention. All patents, patent applications and publications referred to herein are hereby incorporated by reference.

### NUMBERED REFERENCES

1. Muhlestein J B. Chlamydia pneumoniae-induced atherosclerosis in a rabbit model. J Infect Dis 2000;181 Suppl 3: S505-7.
2. Moazed T C, Kuo C C, Patton D L, Grayston J T, Campbell L A. Experimental rabbit models of Chlamydia pneumoniae infection. Am J Pathol 1996;148:667-76.
3. Fong I W, Chiu 13, Viira E, Fong M W, Jang D, Mahoney J. Rabbit model for Chlamydia pneumoniae infection. J Clin Microbiol 1997; 35:48-52.
4. Moazed T C, Kuo C C, Grayston J T, Campbell L A. Murine models of Chlamydia pneumoniae infection and atherosclerosis. J Infect Dis 1997; 175:883-90.
5. Hu H. et al. (1999) The atherogenic effects of chlamydia are dependent on serum cholesterol and specific to Chlamydia pneumoniae. J. Clin. Invest. 103:747-53.
6. Liu L. et al. (2000) Chlamydia pneumoniae infection significantly exacerbates aortic atherosclerosis in an LDLR−/−mouse model within six months. Mol Cell Biochem 215:123-8.
7. Gurfinkel E, Bozovich G, Daroca A, Beck E, Mautner B. Randomised trial of roxithromycin in non-Q-wave coronary syndromes: ROXIS pilot study. Lancet 1997; 350:404-7.
8. Gupta S, Leatham E W, Carrington D, Mendall M A, Kaski J C, Camm A J. Elevated Chlamydia pneumoniae antibodies, cardiovascular events, and azithromycin in male survivors of myocardial infarction. Circulation 1997; 96:404-7.
9. Blasi R. Denti F, Erba M, et al. Detection of Chlamydia pneumoniae but not Helicobacter pylori in atherosclerotic plaques of aortic aneurysms. J Clin Microbiol 1996; 34:2766-9.
10. Blanchard J F, Armenian H K, Peeling R, Poulter Friesen P, Shen C, Brunham RC. The relation between Chlamydia pneumoniae infection and abdominal aortic aneurysm: a case-control study. Clin Infect Dis 2000; 30:946-7.
11. Grayston JT. Background and current knowledge of Chlamydia pneumoniae and atherosclerosis. J Infect Dis 2000; 181 Suppl 3: S402-10.
12. Capron L. Chlamydia in coronary plaques—hidden culprit or harmless hobo? Nat Med 1996; 2:856-7.
13. Haberbosch W, Jantos C. Chlamydia pneumoniae infection is not an independent risk factor for arterial disease. Herz 2000; 25:79-83.
14. Peeling R W, Kimani J, Plummer F. Antibody to chamydial hsp60 predicts an increased risk for chlamydial pelvic inflammatory disease. J Infect Dis 1997; 175:1153-8.
15. Kalman S, Mitchel W, Marathe R, et al. Comparative genomes of Chlamydia pneumoniae and C. trachomatis. Nat Genet 1999; 21:285-9
16. Read T D, Brunham R C, Shen C, et al. Genome sequences of Chlamydia trachomatis MoPn and Chlamydia pneumoniae AR39. Nucleic Acids Res 2000; 28:1397-406.
17. Shira M., Hirakawa H, Kimoto M, et al. Comparison of whole genome sequences of Chlamydia pneumoniae J138 from Japan and CWL029 from USA. Nucleic Acids Res 2000; 28:2311-4
18. Boyd E F, Davis B M, Hochhut B. Bacteriophage-bacteriophage interactions in the evolution of pathogenic bacteria. Trends Microbiol 2001; 9:137-44.
19. Pham, B. G. et al. (1998) J. Clin. Microbiol. 36:1919-1922
20. Blanchard J F, Armenian H, Poulter Friesen P. Risk factors for abdominal aortic aneurysms: a case-control study. Am J Epidemiol 2000; 151:575-83.
21. Zwiebel W J. Aortic and iliac aneurysm. Sem Ultrasound CT MRI 1992; 13:69-80.
22. Cohen C R, Nguti R, Bukusi E A, et al. Human immunodeficiency virus type 1-infected women exhibit reduced interferon-gamma secretion after Chlamydia trachomatis stimulation of peripheral blood lymphocytes. J Infect Dis 2000; 182:1672-7.
23. Watson, M W et al. (1994) Microbiology 140:2003-11.

1. A method for determining whether a subject is susceptible to vascular disease, comprising
testing a biological sample from said subject for the presence or absence of bacteriophage ΦCpn1, a component of a host Chlamydia bacterium or an antibody to said bacteriophage or said Chlamydia component,
wherein detection of the presence of said bacteriophage. Chlamydia component or antibody is indicative of the presence of said host Chlamydia bacteria in the subject and susceptibility of the subject to vascular disease.

2. (cancel)

3. The method of claim 1, wherein the sample is a bodily fluid from the subject to be tested, and the method further comprises the step of obtaining the bodily fluid.

4. The method of claim 1, wherein the sample is a fraction of a bodily fluid from said subject, which fraction contains bacteria, virus, antibody, peptide or nucleic acid.

5. The method of claim 1, wherein the host Chlamydia is C. pneumoniae.

6. The method of claim 5, wherein the C. pneumoniae is of the strain AR39.

7. The method of claim 1, wherein detection of the host Chlamydia component being detected comprises a Chlamydia elementary body.

8. The method of claim 7, wherein the presence of said elementary body is detected by detecting in the sample an antibody to said Chlamydia elementary body.

9. The method of claim 7, wherein the presence of said elementary body is detected by measuring antibody binding to the elementary body.

10. The method of claim 1, wherein at the time of testing, it is unknown whether the subject has a risk factor or predisposition to vascular disease.

11. The method of claim 1, wherein the subject does not have a hypercholesterolemic condition.

12. The method of claim 1, wherein detection of the bacteriophage host comprises detecting one or more of:
(a) a ΦCpn1 peptide
(b) a peptide having an amino acid sequence substantially identical to a ΦCpn1 peptide, or
(c) an antibody to the ΦCpn1 peptide.

13. The method of claim 12 that measures antibody binding to the peptide.

14. The method of claim 13 that measures, antibody binding to a ΦCpn1 peptide.

15. The method of claim 14, wherein the ΦCpn1 peptide is a ΦCpn1 capsid peptide.

16. The method of claim 13 that measures binding of an antibody that is capable of binding to a polypeptide or peptide having the sequence of any one of SEQ ID NO:8- SEQ ID NO:13.

17. The method of claim 16 that measures binding of an antibody that is capable of binding to Vp1 (SEQ ID NO:8).

18. The method of claim 12, wherein the antibody being detected is present in the sample.

19. The method of claim 12, wherein the detecting comprises sequencing peptides in the sample and comparing sequences so obtained to the sequences designated as SEQ ID NO:8- EQ ID NO:13, to determine whether peptides in the sample comprise
(i) one or more of SEQ ID NO:8- SEQ ID NO: 13, or
(ii) a sequence substantially identical to one or more of SEQ ID NO:8- SEQ ID NO:13.

20. The method of claim 1, wherein detection of the bacteriophage comprises detecting a ΦCpn1 nucleic acid, or a nucleic acid having a sequence substantially identical to a ΦCpn1 nucleic acid.

21. The method of claim 20, further comprising the step of amplifying ΦCpn1 nucleic acids in the sample.

22. The method of claim 20, further comprising the step or steps of recombinantly expressing ΦCpn1 nucleic acids in the sample to provide the nucleic acid.

23. The method of claim 20, wherein the detecting comprises measuring hybridization of a complementary oligonucleotide to the nucleic acid.

24. The method of claim 20, wherein the detecting comprises sequencing of nucleic acids in the sample and comparing sequences so obtained to SEQ ID NO: 1.

25. The method of claim 1, further comprising after determining the presence of said bacteriophage, Chlamydia component or antibody, the additional step of selecting a course of treatment for said subject's Chlamydia infection.

26. The method of claim 1, further comprising treating the subject for Chlamydia infection.

27. The method of claim 26, wherein the treatment comprises administering to the subject an antibiotic effective against Chlamydia.

28. An isolated antibody that binds to a bacteriophage ΦCpn1 polypeptide or peptide having a sequence SEQ ID NO:8, SEQ ID NO:9, SEQ ID NO:10, SEQ ID NO:11, SEQ ID NO:12 or SEQ ID NO:13.

29. The antibody of claim 28, produced by a method which comprises:
(a) immunizing a mammal with a polypeptide or peptide comprising one of more of:
(i) SEQ ID NO:8.
(ii) SEQ ID NO:9,
(iii) SEQ ID NO:10,
(iv) SEQ ID NO: 11,
(v) SEQ ID NO:12,
(vi) SEQ ID NO:13,
(vii) an immunogenic fragment of any of (i)-(vi), or
(viii) a polypeptide or peptide having substantial sequence identity to any of (i)-(vi); and,
(b) isolating said antibody or a cell producing said antibody from the mammal.

30. The antibody of claim 28, that binds to a ΦCpn1 capsid protein.

31. The antibody of claim 28, that binds to Vp1 (SEQ ID NO:8).

32. An immunogenic composition for eliciting an immune response in a mammal against bacteriophage ΦCpn1 or against Chlamydia bacteria that comprise bacteriophage ΦCpn1, the composition comprising
(A) a pharmaceutically acceptable carrier and
(B) an immunogen which comprises:
(i) one or more polypeptides or peptides having a sequence (a) SEQ ID NO:8, (b) SEQ ID NO:9, (c) SEQ ID NO:10, (d) SEQ ID NO:11, (e) SEQ ID NO:12, or (f) SEQ ID NO:13,
(ii) an immunogenic fragment of any of (a)-(f),
(iii) a polypeptide or peptide having substantial sequence identity to any of (a)-(f); or
(iv) a nucleic acid molecule encoding any one or more of said polypeptides, peptides or fragments of (i), (ii) or (iii).

33. The composition of claim 32 wherein the immunogen is one of more of said polypeptides or peptides of (i).

34. A kit for use in a method for testing a biological sample for the presence of a bacteriophage ΦCpn1, a component of a chlamydia host of said bacteriophage, or an antibody to said bacteriophage or said Chlamydia component, which kit is useful for determining whether a subject is susceptible to vascular disease,
the kit comprising, in a commercial package:
(i) one or more detecting moieties selected from the group consisting of:
(a) a ΦCpn1 polypeptide or a peptide fragment thereof;
(b) a peptide having substantial sequence identity to the ΦCpn1 polypeptide or peptide fragment of (a);
(c) an antibody that binds to a ΦCpn1 peptide;
(d) one or more oligonucleotides complementary to a ΦCpn1 nucleic acid;
(e) an antibody that binds to a Chlamydia AR39 elementary body; and
(f) a peptide that binds to the elementary body-binding antibody of (e);
and
(ii) instructions for use of the one or more detecting moieties for detecting the presence of said bacteriophage ΦCpn1 or Chlamydia host component in said sample.

35. The kit of claim 34, wherein the detecting moiety is an antibody that binds to a ΦCpn1 peptide.

36. The kit of claim 35, further comprising a peptide that binds to the ΦCpn1 peptide-binding antibody.

37. The kit of claim 34, wherein the detecting moiety is said ΦCpn1 polypeptide, said peptide fragment, or said peptide that has substantial sequence identity to the ΦCpn1 polypeptide or peptide fragment thereof.

38. The kit of claim 37, further comprising an antibody that binds to an immunoglobulin molecules of the subject from whom the sample is derived.

39. The method of 25 further comprising treating the subject for Chlamydia infection in accordance with the selected course of treatment.

40. The method of claim 39, wherein the treatment comprises administering to the subject an antibiotic effective against Chlamydia.


SEQUENCE LISTING (21 sequences)

SEQ ID NO: 1 - DNA, 4532 bp, phage PHI Cpn1 of Chlamydia pneumoniae AR39
gatccttcgaaaaatcaatttgccgcgcatcctgaagattacattctctatgagattggatcttacgatgactctactggaactttcattcccttagatgtgcctaaagccttaggaacaggcttggattttaagcacaaacagtagggaagatatggttaggaatcggcgtttgccttcagttatgagtcattctttcgcgcaagttccatcagcgcgaattcagagaagttcttttgatagatcttgtggtttaaagactacattcgacgccggttacctaatccctatcttttgcgatgaagttctccctggagatactttctccttgaaagaggcgtttttagcacgtatggctacgcctatctttcctcttatggataatttgcgtttagatacgcagtatttctttgttcctcttcgacttatatggtcgaatttccaaaagttctgtggagaacaagataatcctggagattccacagattttcttaccccagttttaaccgctcctagtgggggatttactgaaggatcaatccatgattatcttggtctacctactaaagttgcaggaattgaatgtgttgcgttctggcacagagcttacaatttgatttggaaccagtactatcgtgatgagaatattcaggaatctgttgacgtggaaatgggagacaccacctctaatgaggtgaataattataagcttcttaagcgtgggaagcgttatgattacttcacttcatgtctcccttggccacaaaaaggtcctgcagtgacaatcggagttggaggtattgttcctgttcaaggtttaggaattcaatgggggaattctagtgctccaaatcctataactgcttctagttggataaattctgttaatcctacatttataaattctacaacgccgacgcctacaggaacgaatcagattttgaattatggtcaggcgtattatattaagaagcctggagaagcaactacagatcctacacctagggcttatgtagatttaggttctacttctcctgtgacgattaattctcttcgtgaagctttccaattgcaaaagctttatgagagagatgctcgtggcggaacaaggtacattgagattattcgttcccatttcaatgtgcagtctccagatgcaaggttgcaacgtgcagagtatcttggaggttcttcaactcctgtgaatatttctccgattccacagacttcctcaacagactccacatctcctcaaggaaatcttgctgcttatggtacagcgattggatcgaagcgagtcttcacaaagtctttcacagaacatggtgtaattcttggattagcctctgtacgcgccgatctcaactatcagcaaggtttggataggatgtggtcacgaagaacgcgctgggacttttactggcctgctcttagccatttaggtgagcaagctgtgctcaataaagagatctattgccaaggtcctgcagttaaggatgctcagaatggtaatgttgttgtggatgagcaagtctttggatatcaggagagatttgcggagtatcgctataagacttcgaaaatcactgggaagttccgatcaaatgctacaggttctttagatgcatggcatttagctcagcagtttgagaatcttccaacactttctccagagtttatcgaagaaaatcctcctatggatcgtgttgttgctgtagatactgagccagattttctcttagatggttggttttcattgcgttgtgcaagaccaatgcctgtctattctgttccaggtcttattgatcatttctaatttccactcagttttccggtttgataaagcaaactcacgttcgtagataagtgagtacggtgaaaaccaaaacggaaagctgaggcgtaaaaatgtggagaatttatgaatcccgaacaactcacgagtactctcggttcagcagtttctggagttgcccaaggattatccttcctccctggaatagcttccggagttttaggatatcttggcgcacagaaacaaaatgcgactgcgaagcaaattgctagagagcaaatggcttttcaggagcgcatgtctaacacggcataccaacgtgctatggaagacatgaagaaagctggccttaaccctatgttagctttttctaaaggcggtgcttcttctcctgcaggagcgtcatggtctccgaataatcctgtagagagcgcgatgaattctggacttgccgtgcaaagacttacttatgaacgtaagaaaatgcaggcagagcttcagaatcttcgtgagcagaaccgtttgattagaaaccaagcaatacgtgaaggctatctcgcagaacgagataaatatatgcgtgttgctggagttcctgtggctactgagatgttagatagaacttcaggtcttctctcatcttcagctaaggcatttaagaatcttttttcaagaaaaggaaggtagatgtttaagtcggcatattccgaaaaaaaatctgtaaagatgaagtttacacagaaatctttgacgcaacaacacaacaaagatgagtgtgatattaacaacattgtcgcaaaactcaacgctacaggcgttttagagcacgtagagcgacgatctccacgttatatggactgtatggaccctatggagtattccgaggctctaaacgtcgttattgaggctcaggagcaatttgactctttaccagcgaaagttcgtgaacgttttggaaatgatccggaagcgatgctcgatttcttgagccgtgaagaaaattatgaagaagcaaaggcgttaggttttgtttatgaagatggaacttctggagcacctcaaacattttttgaagctgatcctaaagatgatcaaaatgtggcaaaccaagaacctggattagcccaaaaatgagcaaattttgtgcaaaaaagtgtgcaaaaaatgcccaaaaaaagggccaaaaaatgcccccaaaatcggagcattttacgagagaaaaacacctagacttaaacagtctacttgatctgtttaagtctaggtgataccgattcaaggaaatatttgaaaaaataagccctatttagggcccaaaatttaagcttaaaatgaggttaaaaatggcacgaagaagatacagacttccgcgacgtagaagtcgaagacttttttcaagaactgcattaaggatgcatccaagaaataggcttcgaagaattatgcgtggcggcattaggttctagttttggacgttaaggaaatctttaaggttatgctaaattaactgtcatgtataatttggctcgtgaccgaatagtcatattcgcactgaataaattgtacacaacagttgaaggcttggatgttgatttttaatgtcttagccttcatttttggtttgttgtaactttaatttaattcaggcatttatggcgtgtgtctctcctttcgtatgttttatagattcttataaccagctctggtttcccaaaggtaagaagtctcctaaaccttgggataaagtccgtgaattaaattcttttgagcaaactcaacctgaagagtatcgaaaacgatgggttgtgatgccttgtcttaagtgtaggttttgcagagtgcggaatgcaaagatttggtcgtatcgttgcatgcacgaagcgtctttatattctcagaattgttttttaactttgacttatgaggatcgttatcttccagagaatggctctttggtgagaaatcatcctcgtttgtttcttatgcgattgaggaaagagatctatcctcataaaattcgttattttggatgtggtgaatatggatcgaaattacaaaggcctcattatcatcttcttatttataattacgattttcctgataagaagctcttgagtaaaaagcgtggcaatcctctctttgtttctgagaagttaatgcggctttggccttttggattctctacagtaggatctgtaacgcggcagagtgcaggttatgtagcgcgatattctttgaagaaagtgaatggagatatttctcaagatcattacggtcaaagacttccggagtttcttatgtgttctcttaaaccaggaataggagcggattggtatgagaaatataaacgcgatgtttatcctcaggattatcttgttgtgcaagataaagggaagtcttttaagacgcgtcctccacgttactatgataagttacattctcggtttgatccggaagagatggaagagatcaaacaaaaacgcgtagagaaatttatggctttgcctgagttaactcaagataaggctgaggtgaagcaatatattttcaatgaccgtacgaagagactctttagagactatgaggaggagagttactaaacttttttaaaaaataggagcttttttcaatgaaagtttttacagtgtttgatattaagacggaaatttatcagcagcctttctttatgcaggctacgggagcggcaattagagcgttttccgatatggtaaatgag

SEQ ID NO: 2 - DNA, 1659 bp, phage PHI Cpn1 of Chlamydia pneumoniae AR39
atggttaggaatcggcgtttgccttcagttatgagtcattctttcgcgcaagttccatcagcgcgaattcagagaagttcttttgatagatcttgtggtttaaagactacattcgacgccggttacctaatccctatcttttgcgatgaagttctccctggagatactttctccttgaaagaggcgtttttagcacgtatggctacgcctatctttcctcttatggataatttgcgtttagatacgcagtatttctttgttcctcttcgacttatatggtcgaatttccaaaagttctgtggagaacaagataatcctggagattccacagattttcttaccccagttttaaccgctcctagtgggggatttactgaaggatcaatccatgattatcttggtctacctactaaagttgcaggaattgaatgtgttgcgttctggcacagagcttacaatttgatttggaaccagtactatcgtgatgagaatattcaggaatctgttgacgtggaaatgggagacaccacctctaatgaggtgaataattataagcttcttaagcgtgggaagcgttatgattacttcacttcatgtctcccttggccacaaaaaggtcctgcagtgacaatcggagttggaggtattgttcctgttcaaggtttaggaattcaatgggggaattctagtgctccaaatcctataactgcttctagttggataaattctgttaatcctacatttataaattctacaacgccgacgcctacaggaacgaatcagattttgaattatggtcaggcgtattatattaagaagcctggagaagcaactacagatcctacacctagggcttatgtagatttaggttctacttctcctgtgacgattaattctcttcgtgaagctttccaattgcaaaagctttatgagagagatgctcgtggcggaacaaggtacattgagattattcgttcccatttcaatgtgcagtctccagatgcaaggttgcaacgtgcagagtatcttggaggttcttcaactcctgtgaatatttctccgattccacagacttcctcaacagactccacatctcctcaaggaaatcttgctgcttatggtacagcgattggatcgaagcgagtcttcacaaagtctttcacagaacatggtgtaattcttggattagcctctgtacgcgccgatctcaactatcagcaaggtttggataggatgtggtcacgaagaacgcgctgggacttttactggcctgctcttagccatttaggtgagcaagctgtgctcaataaagagatctattgccaaggtcctgcagttaaggatgctcagaatggtaatgttgttgtggatgagcaagtctttggatatcaggagagatttgcggagtatcgctataagacttcgaaaatcactgggaagttccgatcaaatgctacaggttctttagatgcatggcatttagctcagcagtttgagaatcttccaacactttctccagagtttatcgaagaaaatcctcctatggatcgtgttgttgctgtagatactgagccagattttctcttagatggttggttttcattgcgttgtgcaagaccaatgcctgtctattctgttccaggtcttattgatcatttc

SEQ ID NO: 3 - DNA, 558 bp, phage PHI Cpn1 of Chlamydia pneumoniae AR39
atgaatcccgaacaactcacgagtactctcggttcagcagtttctggagttgcccaaggattatccttcctccctggaatagcttccggagttttaggatatcttggcgcacagaaacaaaatgcgactgcgaagcaaattgctagagagcaaatggcttttcaggagcgcatgtctaacacggcataccaacgtgctatggaagacatgaagaaagctggccttaaccctatgttagctttttctaaaggcggtgcttcttctcctgcaggagcgtcatggtctccgaataatcctgtagagagcgcgatgaattctggacttgccgtgcaaagacttacttatgaacgtaagaaaatgcaggcagagcttcagaatcttcgtgagcagaaccgtttgattagaaaccaagcaatacgtgaaggctatctcgcagaacgagataaatatatgcgtgttgctggagttcctgtggctactgagatgttagatagaacttcaggtcttctctcatcttcagctaaggcatttaagaatcttttttcaagaaaaggaagg

SEQ ID NO: 4 - DNA, 444 bp, phage PHI Cpn1 of Chlamydia pneumoniae AR39
atgtttaagtcggcatattccgaaaaaaaatctgtaaagatgaagtttacacagaaatctttgacgcaacaacacaacaaagatgagtgtgatattaacaacattgtcgcaaaactcaacgctacaggcgttttagagcacgtagagcgacgatctccacgttatatggactgtatggaccctatggagtattccgaggctctaaacgtcgttattgaggctcaggagcaatttgactctttaccagcgaaagttcgtgaacgttttggaaatgatccggaagcgatgctcgatttcttgagccgtgaagaaaattatgaagaagcaaaggcgttaggttttgtttatgaagatggaacttctggagcacctcaaacattttttgaagctgatcctaaagatgatcaaaatgtggcaaaccaagaacctggattagcccaaaaa

SEQ ID NO: 5 - DNA, 105 bp, phage PHI Cpn1 of Chlamydia pneumoniae AR39
ttgtgcaaaaaagtgtgcaaaaaatgcccaaaaaaagggccaaaaaatgcccccaaaatcggagcattttacgagagaaaaacacctagacttaaacagtctact

SEQ ID NO: 6 - DNA, 93 bp, phage PHI Cpn1 of Chlamydia pneumoniae AR39
ttggatgcatccttaatgcagttcttgaaaaaagtcttcgacttctacgtcgcggaagtctgtatcttcttcgtgccatttttaacctcattt

SEQ ID NO: 7 - DNA, 981 bp, phage PHI Cpn1 of Chlamydia pneumoniae AR39
ttggtttgttgtaactttaatttaattcaggcatttatggcgtgtgtctctcctttcgtatgttttatagattcttataaccagctctggtttcccaaaggtaagaagtctcctaaaccttgggataaagtccgtgaattaaattcttttgagcaaactcaacctgaagagtatcgaaaacgatgggttgtgatgccttgtcttaagtgtaggttttgcagagtgcggaatgcaaagatttggtcgtatcgttgcatgcacgaagcgtctttatattctcagaattgttttttaactttgacttatgaggatcgttatcttccagagaatggctctttggtgagaaatcatcctcgtttgtttcttatgcgattgaggaaagagatctatcctcataaaattcgttattttggatgtggtgaatatggatcgaaattacaaaggcctcattatcatcttcttatttataattacgattttcctgataagaagctcttgagtaaaaagcgtggcaatcctctctttgtttctgagaagttaatgcggctttggccttttggattctctacagtaggatctgtaacgcggcagagtgcaggttatgtagcgcgatattctttgaagaaagtgaatggagatatttctcaagatcattacggtcaaagacttccggagtttcttatgtgttctcttaaaccaggaataggagcggattggtatgagaaatataaacgcgatgtttatcctcaggattatcttgttgtgcaagataaagggaagtcttttaagacgcgtcctccacgttactatgataagttacattctcggtttgatccggaagagatggaagagatcaaacaaaaacgcgtagagaaatttatggctttgcctgagttaactcaagataaggctgaggtgaagcaatatattttcaatgaccgtacgaagagactctttagagactatgaggaggagagttac

SEQ ID NO: 8 - protein, 553 aa, phage PHI Cpn1 of Chlamydia pneumoniae AR39
MVRNRRLPSVMSHSFAQVPSARIQRSSFDRSCGLKTTFDAGYLIPIFCDEVLPGDTFSLKEAFLARMATPIFPLMDNLRLDTQYFFVPLRLIWSNFQKFCGEQDNPGDSTDFLTPVLTAPSGGFTEGSIHDYLGLPTKVAGIECVAFWHRAYNLIWNQYYRDENIQESVDVEMGDTTSNEVNNYKLLKRGKRYDYFTSCLPWPQKGPAVTIGVGGIVPVQGLGIQWGNSSAPNPITASSWINSVNPTFINSTTPTPTGTNQILNYGQAYYIKKPGEATTDPTPRAYVDLGSTSPVTINSLREAFQLQKLYERDARGGTRYIEIIRSHFNVQSPDARLQRAEYLGGSSTPVNISPIPQTSSTDSTSPQGNLAAYGTAIGSKRVFTKSFTEHGVILGLASVRADLNYQQGLDRMWSRRTRWDFYWPALSHLGEQAVLNKEIYCQGPAVKDAQNGNVVVDEQVFGYQERFAEYRYKTSKITGKFRSNATGSLDAWHLAQQFENLPTLSPEFIEENPPMDRVVAVDTEPDFLLDGWFSLRCARPMPVYSVPGLIDHF

SEQ ID NO: 9 - protein, 186 aa, phage PHI Cpn1 of Chlamydia pneumoniae AR39
MNPEQLTSTLGSAVSGVAQGLSFLPGIASGVLGYLGAQKQNATAKQIAREQMAFQERMSNTAYQRAMEDMKKAGLNPMLAFSKGGASSPAGASWSPNNPVESAMNSGLAVQRLTYERKKMQAELQNLREQNRLIRNQAIREGYLAERDKYMRVAGVPVATEMLDRTSGLLSSSAKAFKNLFSRKGR

SEQ ID NO: 10 - protein, 148 aa, phage PHI Cpn1 of Chlamydia pneumoniae AR39
MFKSAYSEKKSVKMKFTQKSLTQQHNKDECDINNIVAKLNATGVLEHVERRSPRYMDCMDPMEYSEALNVVIEAQEQFDSLPAKVRERFGNDPEAMLDFLSREENYEEAKALGFVYEDGTSGAPQTFFEADPKDDQNVANQEPGLAQK

SEQ ID NO: 11 - protein, 35 aa, phage PHI Cpn1 of Chlamydia pneumoniae AR39
MCKKVCKKCPKKGPKNAPKIGAFYERKTPRLKQST

SEQ ID NO: 12 - protein, 31 aa, phage PHI Cpn1 of Chlamydia pneumoniae AR39
MDASLMQFLKKVFDFYVAEVCIFFVPFLTSF

SEQ ID NO: 13 - protein, 327 aa, phage PHI Cpn1 of Chlamydia pneumoniae AR39
MVCCNFNLIQAFMACVSPFVCFIDSYNQLWFPKGKKSPKPWDKVRELNSFEQTQPEEYRKRWVVMPCLKCRFCRVRNAKIWSYRCMHEASLYSQNCFLTLTYEDRYLPENGSLVRNHPRLFLMRLRKEIYPHKIRYFGCGEYGSKLQRPHYHLLIYNYDFPDKKLLSKKRGNPLFVSEKLMRLWPFGFSTVGSVTRQSAGYVARYSLKKVNGDISQDHYGQRLPEFLMCSLKPGIGADWYEKYKRDVYPQDYLVVQDKGKSFKTRPPRYYDKLHSRFDPEEMEEIKQKRVEKFMALPELTQDKAEVKQYIFNDRTKRLFRDYEEESY

SEQ ID NO: 14 - DNA, 24 bp, Artificial Sequence
  oligonucleotide primer for the vp1 gene forward
cgctcctagtgggggatttactga

SEQ ID NO: 15 - DNA, 24 bp, Artificial Sequence
  oligonucleotide primer for the vp1 gene reverse
cacagcttgctcacctaaatggct

SEQ ID NO: 16 - DNA, 22 bp, Artificial Sequence
  oligonucleotide primer for the 16s RNA gene forward
cggtaatacggagggtgctagc

SEQ ID NO: 17 - DNA, 24 bp, Artificial Sequence
  oligonucleotide primer for the 16s gene reverse
gaattaaaccacatgctccactgc

SEQ ID NO: 18 - DNA, 21 bp, Artificial Sequence
  oligonucleotide primer for the C. pneumoniae CP0543 gene forward
ctgtaaggtgaaaagttttta

SEQ ID NO: 19 - DNA, 19 bp, Artificial Sequence
  oligonucleotide primer for the C. pneumoniae CP0543 gene reverse
cagctgtaaatgcagcttt

SEQ ID NO: 20 - DNA, 21 bp, Artificial Sequence
  forward primer containing restriction site
agtagggaagccatggttagg

SEQ ID NO: 21 - DNA, 27 bp, Artificial Sequence
  reverse primer containing restriction site
actgactcgagattagaaatgatcaat